(19) United States
(12) Patent Application Publication
(10) Pub. No.: US 2003/0148360 A1
(43) Pub. Date: Aug. 7, 2003

(54) Replicable probe array

(75) Inventors: Patrick E. Guire, Eden Prairie, MN (US); Melvin J. Swanson, Carver, MN (US)

Correspondence Address: MERCHANT & GOULD PC, P.O. BOX 2903, MINNEAPOLIS, MN, 55402-0903, US

(73) Assignee: SurModics, Inc., Eden Prairie, MN (US)

(21) Appl. No.: 10/346,691

(22) Filed: Jan. 15, 2003

Related U.S. Application Data
(63) Continuation of application No. 09/631,139, filed on Aug. 2, 2000, now abandoned.
Application No. 09/631,139 is a continuation-in-part of application No. 09/240,466, filed on Jan. 29, 1999, now Pat. No. 6,514,768.

(51) Int. Cl. C12Q 1/68; G01N 33/53; C12M 1/34; G01N 33/543

(52) U.S. Cl. 435/6; 435/7.1; 435/287.2; 436/518

(57) ABSTRACT

A system for producing substantially identical specific binding ligand probe arrays, for instance, by preparing and replicating an original master array and/or by providing a reusable assay array that is capable of being regenerated. In one embodiment the system includes the preparation and use of a) a master array surface having address ligands immobilized thereon, b) a multi-ligand conjugate having a binding domain complementary to an address ligand, a binding domain complementary to a target ligand, and a third ligand for use in transferring the conjugates into or onto the surface of assay array, which can be used with or upon disassociation of the address and its complementary ligands.

This is a Continuation-in-Part of U.S. patent application Ser. No. 09/240,466 filed Jan. 29, 1999.

### TECHNICAL FIELD

The present invention relates to the immobilization of specific binding ligands, such as nucleic acids and other ligands, in a known spatial arrangement. In another aspect, the invention relates to solid supports, such as oligonucleotide arrays, incorporating such nucleic acids. In yet another aspect, the invention relates to photoreactive groups, to molecules and/or surfaces derivatized with such groups, and to the attachment of such molecules to support surfaces by the activation of such groups.

### BACKGROUND OF THE INVENTION

The development of oligonucleotide probe arrays, more commonly known as “DNA chips” and “Gene Chip” (a registered trademark of Affymetrix, Inc.), has made significant advances over the past few years, and is becoming the center of ever-increasing attention and heightened significance. See, for instance, Stipp, D., Fortune, p.56, Mar. 31, 1997. See also Borman, S., C&EN, p.42, Dec. 9, 1996, and Travis, J., Science News 151:144-145 (1997). These 2- or 3-cm square chips are capable of containing tens of thousands to hundreds of thousands of immobilized oligonucleotides, allowing researchers to witness for the first time the behavior of thousands of genes acting in concert. DNA chips are useful for observing unique gene expression patterns, gauging the success of drug treatment, tailoring medications to patients based upon their genetic makeup, sequencing genes, and conducting research in the area of genetic medicine. See also, “Microchip Arrays Put DNA on the Spot”, R. Service, Science 282(5388):396-399, Oct. 16, 1998; and “Fomenting a Revolution, in Miniature”, I. Amato, Science 282(5388): 402-405, Oct. 16, 1998.

Typically, oligonucleotide probe arrays display specific oligonucleotide sequences at precise locations in an information rich format. In use, the hybridization pattern of a fluorescently labeled nucleic acid target is used to gain primary structure information for the target. This format can be applied to a broad range of nucleic acid sequence analysis problems including pathogen identification, forensic applications, monitoring mRNA expression and de novo sequencing. See, for instance, Lipshutz, R. J., et al., BioTechniques 19(3):442-447 (1995). Such arrays sometimes need to carry several tens of thousands, or even hundreds of thousands of individual probes. The chips also need to provide a broad range of sensitivities in order to detect sequences that can be expressed at levels anywhere from 1 to 10,000 copies per cell.

A variety of approaches have been developed for the fabrication and/or use of oligonucleotide probe arrays. See, for instance, Weaver, et al. (WO 92/10092) which describes a synthetic strategy for the creation of large scale chemical diversity on a solid-phase support. The system employs solid-phase chemistry, photolabile protecting groups and photolithography to achieve light-directed, spatially addressable, parallel chemical synthesis. Using the proper sequence of masks and chemical stepwise reactions, a defined set of oligonucleotides can be constructed, each in a predefined position on the surface of the array.

Using this technology, Affymetrix, Inc. (Santa Clara, Calif.), has developed libraries of unimolecular, double-stranded oligonucleotides on a solid support. See, for instance, U.S. Pat. No. 5,770,722, which describes arrays containing oligonucleotides from 4 to 100 nucleotides in length. The arrays comprise a solid support, an optional spacer, a first oligonucleotide, a second oligonucleotide that is complementary to the first, and a flexible linker or probe. The libraries described are useful for screening for such receptors as proteins, RNA or other molecules which bind double-stranded DNA. Another array developed by Affymetrix is described in U.S. Pat. No. 5,837,832. This reference describes methods for making high-density arrays of oligonucleotide probes on silica chips. The oligonucleotide probes are 9 to 20 nucleotides in length and are synthesized directly on a solid support. The arrays comprise oligonucleotide probes that are complementary to a section of the reference sequence.

Synteni (Palo Alto, Calif.) produces arrays of cDNA by applying polylysine to glass slides, followed by printing cDNA onto the coated slides. The arrays are then exposed to UV light, in order to crosslink the DNA with the polylysine. Unreacted polylysine is then blocked by reaction with succinic anhydride. These arrays, called “Gene Expression Microarrays” (GEM™) are used by labeling mRNA prepared from a normal cell with a fluorescent dye, then labeling mRNA from an abnormal cell with a fluorescent dye of a different color. These two labeled mRNA molecules are simultaneously applied to the microarray, where they competitively bind to the immobilized cDNA molecules. This two color coding technique is used to identify the differences in gene expression between two cell samples. (Heller, R. A., et al., Proc. Natl. Acad. Sci. USA, 94:2150-2155 (1997)).

At least one group, Cantor, et al. (U.S. Pat. No. 5,795,714), describes methods for replicating arrays of probes which are said to be useful for the large scale manufacture of diagnostic aids. The patent includes a method for replicating an array of single-stranded probes on a solid support comprising the steps of:

a) synthesizing an array of nucleic acids each comprising a non-variant sequence of length C at a 3′-terminus and a variable sequence of length R at a 5′-terminus;

b) fixing the array to a first solid support;

c) synthesizing a set of nucleic acids each comprising a sequence complementary to the non-variant sequence;

d) hybridizing the nucleic acids of the set to the array;

e) enzymatically extending the nucleic acids of the set using the variable sequences of the array as templates;

f) denaturing the set of extended nucleic acids; and

g) fixing the denatured nucleic acids of the set to a second solid support to create the replicated array of single-stranded probes.

On a separate subject, the assignee of the present invention has previously described a variety of applications for the use of photochemistry, and in particular, photoreactive groups, e.g., for attaching polymers and other molecules to support surfaces. See, for instance, U.S. Pat. Nos. 4,722,906, 4,979,959, 5,217,492, 5,512,329, 5,563,056, 5,637,460, and 5,714,360 and International Patent Application Nos. PCT/US96/08797 (Virus Inactivating Coatings), PCT/US96/07695 (Capillary Endothelialization), and PCT/US97/05344 (Chain Transfer Agents).

In spite of the various developments to date, there remains a need for methods and reagents that improve the immobilization of nucleic acids onto a variety of support materials, e.g., in order to form oligonucleotide probe arrays. What is clearly needed are new and improved methods and reagents for reproducibly preparing specific binding ligand (e.g., nucleic acid) arrays in a cost-effective and efficient manner, while maintaining an accurate, sensitive product.

### SUMMARY OF THE INVENTION

The invention, in one embodiment, provides a system for producing specific binding ligand (e.g., nucleic acid) probe arrays in a substantially identical spatial arrangement, for instance, by preparing and replicating an original “master” array. In another embodiment, the invention provides a reusable assay array that is capable of being regenerated. The present approach can be contrasted with the traditional approach of separately and individually preparing each probe array anew. Additionally, the invention allows replication of a master array that maintains a spatial arrangement established by the master array. The method of the invention can be adapted for use with conventional arrays, in order to provide replicates thereof, but is preferably used with a master array (and other components) specifically designed for such purposes, in the manner described herein.

In a preferred embodiment, the present invention provides a method and system for reproducibly preparing an assay array, the system comprising:

a) a master array comprising a support surface having a plurality of address ligands immobilized thereon;

b) a plurality of multi-ligand conjugates, each multi-ligand conjugate comprising (i) a core; (ii) at least one molecule of a first ligand binding domain, comprising a ligand selected to bind in a complementary manner to a specific address ligand of the master array, (iii) at least one molecule of a second ligand binding domain, comprising a ligand selected to bind in a complementary manner to a characteristic target ligand, and (iv) at least one molecule of a third ligand, wherein the first ligand binding domain, second ligand binding domain, and third ligand are attached to the core; and

c) an assay array support comprising a support surface and a binding partner for binding the third ligand.

Preferably, the address ligands are immobilized on the master array in the form of a patterned, and optionally random, manner. In a preferred embodiment, the first ligand binding domain and second ligand binding domain of the multi-ligand conjugate comprise nucleic acid sequences. In this embodiment, the second ligand binding domain comprises a nucleic acid sequence that is complementary to a target nucleic acid sequence to be detected in a sample. Optionally, address ligands are immobilized in a random manner, and the precise location of each address ligand is determined after the address ligands are immobilized onto the master array support surface.

Alternatively, the present invention provides a method and system for reproducibly preparing an assay array, the system comprising:

a) a master array comprising a support surface having a plurality of address ligands immobilized thereon;

b) a plurality of multi-ligand conjugates, each multi-ligand conjugate comprising: (i) a core; (ii) at least one molecule of a second ligand binding domain, comprising a ligand selected to bind in a complementary manner to an address ligand and to a characteristic target ligand, and (iii) at least one molecule of a third ligand, wherein the second ligand binding domain and third ligand are attached to the core; and

c) an assay array support comprising a support surface and a binding partner for binding the third ligand.

In this embodiment, the use of a first ligand binding domain is not required. According to this embodiment, the second ligand binding domain is not only complementary to the address ligands of the master array, but is also complementary to a target ligand suspected to be present in a sample. The nucleic acid sequences of the assay arrays produced according to this embodiment are complementary to the master array address ligands. If desired, the assay array produced according to this embodiment can be replicated, to produce a copy of the original master array. Preferably, the third ligand is selected from binding ligands and polymerizable groups.

A corresponding preferred method of the invention, wherein the address and target ligands are both nucleic acid sequences involves the steps of:

a) providing a master array as described above;

b) attaching the multi-ligand conjugates thereto, by allowing their respective first ligand binding domains to hybridize to the complementary address ligands of the master array;

c) bringing the assay array support into sufficient proximity with the master array, under conditions suitable to permit the attached multi-ligand conjugates to attach to the assay array support; and

d) disassociating the hybridized complementary nucleic acid sequences under conditions suitable to permit the assay array support to be recovered and used.

The resulting assay array comprises an assay array support having attached thereto a plurality of multi-ligand conjugates, preferably still having third ligands thereon, present in a pattern established by the master array.

In an alternative embodiment, where the multi-ligand conjugate includes a polymerizable group (e.g., in addition to the third ligand, or in place of the third ligand), the multi-ligand conjugates can be maintained in their oriented positions upon the master array surface by polymerizing those groups in situ, in order to form a polymeric backing sufficient to permit the resultant polymerized layer to be supported and used, e.g., transferred to an assay array support, while retaining the spatial arrangement established by the master array addresses.

In yet another embodiment, wherein the multi-ligand conjugate comprises a second ligand binding domain and a third ligand only (i.e., no first ligand binding domain is present), and the address ligand and target ligand are both nucleic acids, the method includes the steps of:

a) providing a master array as described above;

b) attaching the multi-ligand conjugates thereto, by allowing their respective second ligand binding domains to hybridize to the complementary address ligands of the master array;

c) bringing the assay array support into sufficient proximity with the master array, under conditions suitable to permit the attached multi-ligand conjugates to attach to the assay array support; and

d) disassociating the hybridized complementary nucleic acid sequences under conditions suitable to permit the assay array support to be recovered and used.

Once transferred to an assay array support, the second ligand binding domains, in turn, can be used in a conventional manner to determine the presence, in absolute or relative amount, of one or more target ligands in a sample. The order and arrangement of the second ligand binding domains is predetermined and maintained in the course of the replicating method set forth herein. For instance, the resultant assay array can be used in a conventional manner, e.g., by contacting the array with a sample suspected of containing the target ligands, under conditions suitable to permit any target ligand to be bound and detected. In other aspects, the invention provides a method of using such a system; the various components for use in such a system, including a kit or combination of one or more components; as well as an assay array formed by the method of the invention.

### DETAILED DESCRIPTION

The present invention provides a method and system for reproducibly preparing a specific binding ligand array, such as a nucleic acid array. As used herein, “nucleic acids” include polymeric molecules such as deoxyribonucleic acid (DNA), ribonucleic acid (RNA), peptide nucleic acid (PNA), or any sequence of what are commonly referred to as bases joined by a chemical backbone that have the ability to form base pairs or hybridize with a complementary chemical structure. The term includes oligonucleotide, nucleotide, or polynucleotide sequences, and fragments or portions thereof. The nucleic acid can be provided in any suitable form, e.g., isolated from natural sources, recombinantly produced, or artificially synthesized, may be single- or double-stranded, and may represent the sense or antisense strand. While the invention will be described with particular reference to nucleic acids (and their ability to specifically “bind” via hybridization), it is understood that the invention has applicability to other specific binding ligands as well, such as immunological binding pairs or other ligand/anti-ligand binding pairs.

The term “oligonucleotide,” in turn, will be used interchangeably with the term “nucleic acid” to refer generally to short chain (e.g., less than about 100 nucleotides in length, and typically 20 to 50 nucleotides in length) nucleic acid sequences, e.g., as prepared using techniques presently available in the art, such as solid support nucleic acid synthesis, or using DNA replication, reverse transcription, or the like. The exact size of the oligonucleotide will depend upon many factors, which in turn depend upon the ultimate function or use of the oligonucleotide.

As used herein, “target ligand,” or “target” refers to a ligand, such as a nucleic acid sequence, suspected to be contained in a sample and to be detected and/or quantitated in the method or system of the invention. In one embodiment, the nucleic acid comprises a gene or gene fragment to be detected in a sample.

The term “sample” is used in its broadest sense. The term includes a specimen or culture (e.g., microbiological cultures), as well as biological samples.

As used herein, the terms “complementary” or “complementarity,” when used in reference to polynucleotides (i.e., a sequence of nucleotides such as an oligonucleotide or a target nucleic acid), refer to sequences that are related by the base-pairing rules developed by Watson and Crick. For example, for the sequence “T-G-A” the complementary sequence is “A-C-T.” Complementarity may be “partial,” in which only some of the bases of the nucleic acids are matched according to the base pairing rules. Alternatively, there may be “complete” or “total” complementarity between the nucleic acids. The degree of complementarity between the nucleic acid strands has effects on the efficiency and strength of hybridization between the nucleic acid strands.

The terms “complementary,” or “complementarity,” when used with reference to molecules other than nucleic acids, refers to molecules that are capable of binding with a binding partner, such as molecules that are members of a specific binding pair.

The term “hybridization” is used in reference to the pairing of complementary nucleic acids. Hybridization and the strength of hybridization (i.e., the strength of the association between the nucleic acids) is influenced by such factors as the degree of complementarity between the nucleic acids, stringency of the conditions involved, the melting temperature (Tₘ) of the formed hybrid, and the G:C ratio within the nucleic acids.

As used herein, the term “binding sites” in reference to the assay array refers the area upon which a specific ligand is bound. As described in more detail below, the binding sites preferably contain molecules that are members of a specific binding pair, for forming bonds with the multi-ligand conjugate.

In one embodiment, the present invention provides a master array having immobilized thereon a plurality of address ligands, the pattern of which can be controlled and/or determined by any suitable techniques. Thereafter, a composition comprising a plurality of multi-ligand conjugates is brought into binding proximity with the master array, under conditions suitable to permit a binding domain of the multi-ligand conjugate to bind complementary address ligands on the master array support. In a preferred embodiment, the multi-ligand conjugate includes a third ligand that comprises a member of a specific binding pair. In this embodiment, the third ligand serves to transfer the resultant conjugate complex to the assay array support. The master array containing bound conjugate molecules is brought into binding proximity with an assay array support. The assay array support, in turn, bears binding sites available to bind with the third ligand of the multi-ligand conjugate. The binding sites of the assay array support bind to corresponding binding ligands on the multi-ligand conjugate, thus forming a transitory “sandwich” structure in the form of master array/multi-ligand conjugate/assay array support.

Once the “sandwich” structure has been formed, the base pairing between address ligands of the master array and complementary ligands of the multi-ligand conjugate can then be disassociated, in order to permit the assay array to be removed with the multi-ligand conjugates attached thereto. The conjugates are attached in a spatial relationship that corresponds to the spatial positioning established by their initial binding to the master array surface, thereby permitting the location of target ligands to be maintained and determined. The master array, in turn, can be reused repeatedly and in a similar manner to provide additional assay arrays. In another aspect, the invention provides a plurality of substantially identical assay arrays that are formed by replicating a common master array or sequentially replicating one or more assay arrays.

In yet another aspect, the present invention provides a method and system for directly preparing reusable nucleic acid arrays, without the need to first prepare or replicate a master array, for example, wherein the assay array surface is provided in the form of an optical fiber array. In one such embodiment, each of the fibers is provided with a nucleic acid adapted to hybridize to its complementary nucleic acid in a detectable fashion. Suitable fiber arrays for use in such an embodiment are described, for instance, in K. Michael et al., Analytical Chem. 70(7):1242 (1998), the disclosure of which is incorporated herein by reference.

The assay arrays of this invention, in turn, are preferably adapted to detect a wide variety of nucleic acids in a biological sample. In the course of its use, an assay array can be exposed to a sample suspected of containing one or more target ligands, under conditions suitable to permit the target ligands to hybridize to their corresponding binding domain complement on the assay array. The presence or absence of the target nucleic acid on the assay array can be determined with a chosen signal generation and detection system. Such detection methods are known in the art.

A system of the present invention provides a number of advantages over current methods, including, for instance, an optimal combination of such properties as nucleic acid probe density, ease of use, reproducibility, sensitivity, accuracy, and reduced cost. The present invention can, for instance, provide a higher nucleic acid probe density than commercial approaches that currently rely on photolithography. In one embodiment, such probe density is achieved by the use of micro-beads that are smaller than the wavelength of photolithography light systems. Further, the present invention can provide improved assay specificity, for example, by the use of nucleic acids that are longer (and therefore more specific as to binding) than those currently available through photolithographic or solid phase synthesis.

Moreover, the assay array according to the invention can be used with conventional array readers to provide a variety of target nucleic acid capacities, depending, for instance, upon the area density of the immobilized array. The system of the invention can also provide a significant reduction in the cost of manufacture of high-density array slides. The cost of preparing the master array can itself be reduced, for instance, through the use of patterned deposition and immobilization of address nucleic acid sequences, optionally with the use of micro-beads.

As will be seen from the description herein, the invention provides an improved method for replicating probe arrays that provides a universal “master array” that can be used with a wide variety of multi-ligand conjugates to fabricate any number of replicate assay arrays. The universal nature of the master array is provided by virtue of the address ligands that have no particular specificity for a particular assay array. Rather, the address ligands can be used in connection with any number of specific multi-ligand conjugates to fabricate an unique replicate assay array containing probes for detecting target ligands of interest, or a family of target ligands. In this way, the master array provides a spatial arrangement for the generation of any number of replicate assay arrays.

Master Array

According to the invention, the master array comprises a support surface having a plurality of address ligands immobilized thereon. Address ligands can be directly or indirectly immobilized on the master array support surface. Direct immobilization of address oligonucleotides is achieved by derivatization of the address oligonucleotide, the support surface of the master array, or both. Indirect immobilization involves the use of a linking agent to attach the address oligonucleotide to the surface, as described in more detail below.

The support surface of the master array is fabricated from any suitable material to provide an optimal combination of such desired properties as stability, dimensions, shape, and surface smoothness. Preferred materials should not interfere with nucleic acid hybridization and should not be subject to high amounts of non-specific binding of nucleic acids. Suitable materials include biological or nonbiological, organic or inorganic materials. For example, the master array can be fabricated from any suitable plastic or polymer, silicon, glass, ceramic, or metal, and can be provided in the form of a solid, resin, gel, rigid film, or flexible membrane. Suitable polymers include polystyrene, poly(alkyl)methacrylate, poly(vinylbenzophenone), polycarbonate, polyethylene, polypropylene, polyamide, polyvinylidenefluoride, and the like. Preferred materials include glass and silicon.

Dimensions of the master array are determined based upon such factors as the dimensions of the assay array to be created, and extent of address oligonucleotide diversity desired. Preferably, the master array is provided with planar dimensions of between about 0.5 cm and about 7.5 cm in length, and between about 0.5 cm and about 7.5 cm, preferably between about 1 cm and about 2 cm, in width. Arrays can also be singly or multiply positioned on other supports, such as microscope slides (e.g., having dimensions of about 7.5 cm by about 2.5 cm). One of skill in the art could readily adapt the dimensions of the master array for a particular application, given the teaching herein. The master array can be provided in any suitable configuration, including, for example, capillaries, membranes, wafers, pins or needles. Additionally, as described in more detail below, the master array can be provided in the form of one or more optical fibers.

Address oligonucleotides are attached to the master array support surface directly or via linkers. In one embodiment, address oligonucleotides are directly attached to the support surface by providing and/or derivatizing either the master array surface, the oligonucleotide, or both, with one or more reactive groups. In another embodiment, address oligonucleotides are indirectly attached to the master array support surface through the use of linkers. In this embodiment, the master array support surface is coated with a plurality of linkers, such as linking micro-beads that, in turn, have oligonucleotides attached thereto. Hence, when used in combination with linking micro-beads, the master array support surface is preferably provided with sufficient smoothness such that topographical irregularities are smaller than the radius of the linking micro-bead (e.g., a roughness no greater than 50% the diameter of the linking micro-beads of the master array).

As used herein, the term “address oligonucleotides” will be used to refer to nucleic acid sequences that are immobilized onto the master array to provide a template array for use in the invention. The template is thus an ordered array of address sequences that allow replicate assay arrays to be created using the positional information established by the master array. The address oligonucleotides are selected to be non-complementary with target nucleic acid sequences potentially present in a biological sample to be tested. As a result, the address oligonucleotides are expected to not form base pairs or otherwise hybridize with any target nucleic acids found in a particular biological sample to be tested, thereby avoiding or minimizing the possibility of background or false positive readings. The oligonucleotides are provided in sufficient length to provide suitable diversity of “addresses” on the master array, while at the same time avoiding substantial complementarity to target nucleic acid (e.g., naturally-occurring) sequences to be detected by the assay array. Preferably, these address oligonucleotides are between 10 and 100 nucleotides in length, and most preferably between 20 and 50 nucleotides in length.

As used herein, the term “immobilized” means the address oligonucleotide is attached to the support surface in a sufficiently stable manner for the purposes herein. Such attachment is preferably covalent, although other suitable stable attachment is also contemplated. Suitable attachment will be apparent from the teachings herein.

According to the invention, deposition of the address oligonucleotides onto the master array surface is accomplished in any suitable manner, including for instance, by direct attachment or indirect attachment through a linker (e.g., a linking micro-bead). For example, if the address oligonucleotide is bound to a micro-bead, which is in turn to be bound to the master array, deposition can be accomplished by a gravity-based method in which the specific gravity of the micro-bead causes the individual micro-bead carrying the address oligonucleotide to contact the master array surface. This deposition forms an ordered but random pattern on the master array surface. Preferably, the micro-bead is treated to provide a photoreactive surface functionality. The resultant surface is then illuminated under conditions suitable to activate the photoreactive groups and cause bond formation between the micro-bead carrying address oligonucleotide and the master array support surface.

Preferably, the deposition of the address oligonucleotides is ordered, but random. In this way the address oligonucleotides are placed onto the surface of the master array without first mapping or otherwise determining the exact location of each unique address oligonucleotide. In other words, the address oligonucleotides are deposited on the surface of the master array, are immobilized onto the surface, and their location on the master array is thereafter determined, using known detection techniques. In this way, the invention provides an efficient method of creating a master array that can be used in multiple applications to create assay arrays of any desired composition. For example, once the master array is fabricated and the surface is “mapped” to determine the location of each address oligonucleotide, the master array can be used in connection with any selection of multi-ligand conjugates to prepare an assay array for a particular purpose. This will be apparent through the discussion below.

Direct attachment of the address oligonucleotides to the master array support surface can be accomplished by derivatizing the nucleic acid, the support surface of the master array, or both the nucleic acid and the support surface. Modification of the nucleic acids should preferably avoid substantially modifying the functionalities of the nucleic acid responsible for Watson-Crick base pairing. Preferably, modification of the nucleic acid does not substantially impair the ability of the nucleic acid sequence to hybridize to its complement.

Suitable methods for immobilization of the address oligonucleotide include chemical modification of the oligonucleotide (e.g., amine modification), and non-covalent attachment by high-affinity binding agent (e.g., avidin-biotin). The surface of the master array support, in turn, can be derivatized (e.g., by carboxyl or epoxy groups) in order to link a corresponding reactive group provided by the oligonucleotide itself or a suitable cross-linking reagent. Alternatively, a binding partner (e.g., streptavidin) can be directly attached to the master array support surface, allowing interaction between the address oligonucleotide and the support surface of the master array. Other methods include adsorption of unmodified or modified oligonucleotides, as well as immobilization of the oligonucleotides onto the support surface by ink jet or “needle” printing with thermochemical immobilization of soluble probes on an activated master array support surface. Exemplary embodiments for direct attachment of address oligonucleotides to the master array support surface will now be described, although one of skill in the art will readily recognize that other techniques may be used as well.

Modification of the nucleic acid can be accomplished in any suitable manner to allow attachment to the master array support surface. In one embodiment, the nucleic acid is derivatized to contain at least one reactive moiety that is capable of reacting with the surface of the master array. Alternatively, the nucleic acid is synthesized with a modified base. Other suitable techniques known in the art include modification of the sugar moiety of a nucleotide, or modification of nucleic acid bases, for example, by using N7- or N9-deazapurine nucleosides or the like. Alternatively, the backbone of the nucleic acid can be modified (e.g., phosphoroamidite DNA), so that a reactive group can be attached to the nitrogen center provided by the modified phosphate backbone. One of skill in the art, given the teachings herein, can readily adopt these and other standard nucleic acid modification techniques to attach the nucleic acid sequences to the support surface of the master array.

One embodiment of attachment of photoreactive nucleic acids to surfaces is described, for example, in U.S. patent application Ser. No. 09/028,806 (Guire et al., filed Feb. 24, 1998). This application is commonly owned by the assignee of the present application, and the entire disclosure of this application is incorporated herein by reference. As described in that application, a photoactivatable nucleic acid derivative, in the form of a nucleic acid having one or more photoreactive groups bound thereto, can be provided. The photoreactive groups are preferably covalently bound, directly or indirectly, at one or more points along the nucleic acid. The photoreactive group provides a derivatized nucleic acid that can be selectively and specifically activated in order to attach the nucleic acid to a support, and in a manner that substantially retains its desired chemical or biological function. According to this embodiment, “direct” attachment of the photoreactive group means that the photoreactive compound is attached directly to the nucleic acid. On the other hand, “indirect” attachment refers to attachment of a photoreactive compound and nucleic acid to a common structure, such as a synthetic or natural polymer. The resulting photo-derivatized nucleic acid can be covalently immobilized by the application of suitable irradiation, and usually without the need for surface pretreatment, to a variety of substrate surfaces. The method of this embodiment involves both the thermochemical attachment of one or more photoreactive groups to a nucleic acid and the photochemical immobilization of that nucleic acid derivative upon a substrate surface.

Preferably, oligonucleotides are immobilized onto the master array surface using photoactivatable compounds to form bonds between the address oligonucleotide and the master array support surface. These photoactivatable compounds can be provided in any suitable manner, e.g., by the master array support surface or the address oligonucleotides themselves.

In one embodiment, a reagent composition is used to modify a surface for attachment of a biomolecule, such as a nucleic acid, as described in U.S. patent application Ser. No. 09/227,913 (Chappa et al., filed Jan. 8, 1999), which is commonly owned by the assignee of this invention, and the entire disclosure of which is incorporated herein by reference. As described in that application, a reagent composition is used to covalently attach a target molecule such as a biomolecule (for example, a nucleic acid) which in turn can be used for specific binding reactions (for example, to hybridize a nucleic acid to its complementary strand). The reagent composition comprises one or more thermochemically reactive groups, i.e., groups having a reaction rate dependent on temperature. Suitable groups are selected from the group consisting of activated esters (e.g., N-oxysuccinimide, or “NOS”), epoxide, azlactone, activated hydroxyl and maleimide groups. Optionally, and preferably, the composition further comprises one or more photoreactive groups. Additionally, the reagent may comprise one or more hydrophilic polymers, to which the thermochemically reactive and/or photoreactive groups can be pendent. The photoreactive groups can be used, for instance, to attach reagent molecules to the surface of a support upon the application of a suitable energy source such as light. The thermochemically reactive groups, in turn, can be used to form covalent bonds with appropriate and complementary functional groups on the target molecule.

Generally, according to this embodiment, the reagent molecules are first attached to the surface by activation of the photogroups. Thereafter the oligonucleotide is contacted with the bound reagent under conditions suitable to permit it to come into binding proximity with the bound polymer. The nucleic acid is thermochemically coupled to the bound reagent by reaction between reactive groups of the bound reagent and appropriate functional groups on the nucleic acid molecule. The thermochemically reactive groups can either be on the same polymer or, for instance, on different polymers that are coimmobilized onto the surface. Optionally, and preferably, the target molecule is prepared or provided with functional groups tailored to groups of the reagent molecule. During their synthesis, for instance, the oligonucleotides can be prepared with functional groups such as amines or sulfhydryl groups.

In an alternative embodiment, the surface of the master array is modified with an epoxide-based reagent to allow attachment of the oligonucleotides. One such embodiment is described, for example, in U.S. patent application Ser. No. 09/521,545 (Swan et al., filed Mar. 9, 2000), which patent application is commonly owned by the assignee of the present application, and the entire disclosure of which is incorporated herein by reference. As described in this application, an epoxide-based reagent composition is used for covalent attachment of target molecules onto the surface of a substrate. The method and reagent can be used to covalently immobilize either derivatized or underivatized nucleic acids, and are particularly useful for underivatized nucleic acids. As contemplated in this embodiment, examples of derivatized nucleic acids include amine-derivatized nucleic acids, and underivatized nucleic acids include those not having a group added for the purpose of thermochemical reaction with an epoxide group. The method involves the steps of coating the support with the reagent, printing the nucleic acid array, incubating the slide in a humid environment, blocking excess epoxide groups, and washing the support, after which it is ready for a hybridization assay. In particular, the method comprises the steps of providing a solid support having a surface; providing a reagent comprising one or more epoxide groups, and optionally also comprising one or more photoreactive groups; coating the reagent on the support surface (e.g., covalently attaching the polymeric reagent to the support surface by activation of the photoreactive groups); providing a biopolymer having a corresponding thermochemical reactive group; and attaching the biopolymer to the support by reacting its corresponding reactive group with the bound epoxide group. Optionally, the method further comprises the step of blocking the remaining epoxide groups (e.g., using an amine reagent).

According to this embodiment, the reagent preferably provides one or more epoxide groups (also known as “oxirane” groups) pendent on a polymeric backbone, such as a hydrophilic polyacrylamide backbone. Optionally, the reagent composition further includes pendent photoreactive groups. The photoreactive groups can be used to attach reagent molecules to the surface of the support upon the application of a suitable energy source such as light. The epoxide groups, in turn, can be used to form covalent bonds with appropriate functional groups on the target molecule.

In another embodiment, the support surface of the master array is prepared by uniformly distributing photoreactive bi- or poly-functional reagents on the surface and thereafter coupling these reagents to the surface. In one embodiment, a silicon chip with sufficient smoothness is cleaned and reacted with a photoreactive organosiloxane reagent. This reagent can be prepared, for instance, by bonding a commercial organosiloxane reagent (such as aminopropyldimethylmethoxy siloxane) with a thermochemically reactive photoreagent such as the acid chloride of benzoylbenzoic acid. The resulting photoreactive siloxane reagent is then reacted with the silicon oxide surface of the master array support to provide a smooth surface, primed with photoactivatible groups. The photoreactive groups on the resulting primed surface can be activated and used to coat the oligonucleotides, directly or indirectly by means of linkers, onto the master array support surface.

According to this embodiment, a solution containing a plurality of address oligonucleotides can be applied to the master array support surface. The master array is illuminated with long-wavelength ultraviolet or visible light to photochemically fix the address oligonucleotides onto the master array support surface. In an alternative embodiment, the address oligonucleotides themselves are photoreactive. Suitable photoreactive groups are discussed in more detail below. Optionally, when patterned deposition is desired, a mask or suitable beads could be used. Patterned deposition can be accomplished in any suitable manner. In one embodiment, the oligonucleotides are deposited onto the substrate surface in a patterned array (e.g., using printing techniques), and the substrate can then be uniformly illuminated with light of a suitable wavelength. In another embodiment, oligonucleotides can be uniformly deposited on the substrate surface, followed by patterned illumination (e.g., utilizing masking techniques).

In an alternative embodiment, a melt or concentrated solution of a reagent having both latent reactive (e.g., photoreactive) groups and ligand-attractive (e.g., charged, cationic) groups can be used. For instance, a polystyrene derivative containing one or more of both types of groups (e.g., thiocholine derivatives of polyvinylbenzoic acid) can be deposited on the surface of the master array to provide a reactive surface film for immobilization of the address oligonucleotides. The surface of the master array can be illuminated to generate a smooth photoreactive surface. Optionally, the ligand-attractive group (e.g., cation/thiocholine) is removed, if desired.

Alternatively, the address oligonucleotides are indirectly attached to the master array support surface through the use of linkers or other suitable spacer molecules. “Linker” or “spacer molecule” when used in connection with the immobilization of address oligonucleotides refers to a moiety that is attached to the solid support and is attached to an address oligonucleotide. The linker serves to attach the address oligonucleotides to the master array support surface in a spaced manner to allow the address oligonucleotides to contact the multi-ligand conjugates of the invention.

In one embodiment, the linker is provided in the form of a micro-bead and will be referred to as a “linking micro-bead.” An appropriate bead for use as the linking micro-bead includes any three dimensional structure that is capable of being immobilized onto a solid support as herein described. Preferably, the linking micro-beads are selected to have a specific gravity sufficient to allow them to settle onto the surface of the master array by gravity while avoiding the effects of Brownian motion. Optionally, the micro-bead is porous. The micro-bead can be provided in any suitable size, and nano-beads as well as micro-beads are contemplated in the invention. Linking micro-beads suitable for use in the invention are desirably spherical, and homogeneous in size. Also, preferred beads have a chemical composition, or at least surface, that is subject to bonding with organic reagents. For example, homogeneous silica spheres of between 0.5 micron and about 5 microns (μ) diameter can be reacted with an organosiloxane reagent containing a thermochemically reactive group (e.g., epoxy), in order to couple the reagent to corresponding groups of a binding ligand. The micro-beads can be placed, for instance, by such techniques as electrostatic attraction, electrode or magnetic probes, or optical tweezers.

As contemplated in the invention, the bead can be fabricated from virtually any insoluble or solid material. For example, the bead can be fabricated from silica gel, glass, nylon, resins, Sephadex, Sepharose, cellulose, magnetic beads, Dynabeads, a metal surface (e.g., steel, gold, silver, aluminum, silicon or copper), a plastic material (e.g., polyethylene, polypropylene, polyamide, polyester, polyvinylidenefluoride (PVDF)) and the like, and combinations thereof. Examples of suitable micro-beads are described, for example, in U.S. Pat. No. 5,900,481 (Lough et al., issued May 4, 1999). Other examples of suitable micro-beads are available, for example, from Interfacial Dynamics Corporation (Portland, Oreg.), which provides latex microspheres that can be fluorescent, dyed, and/or provided with surface functionalities as desired for a particular application.

Preferably, the linking micro-beads containing address oligonucleotides are immobilized in a spaced relationship onto the master array support surface. In such an embodiment, a woven screen having spaces only slightly larger than the bead diameter can be used as, or positioned upon, the surface of the master array, in order to promote deposition of micro-beads onto the surface in a separated, but ordered, configuration.

The linking micro-beads can be procured from commercial sources and loaded with oligonucleotides by the use of commercially available processes and materials. The attachment of the address oligonucleotides to the linking micro-bead is preferably accomplished by the formation of covalent bonds between the oligonucleotide and the bead. Methods for attaching address oligonucleotides include those described herein for attachment of address oligonucleotides directly to the master array support surface. In one preferred embodiment, the surface of the linking micro-bead is reacted with an organosiloxane reagent containing a thermochemically reactive group (e.g., an epoxy). Glycidoxypropyltriethoxy siloxane, for instance, can be reacted with the micro-beads to provide an epoxy surface. The 5′ terminus of the address oligonucleotide is, in turn, modified to produce a terminus containing alkylamine groups on spacers. The epoxy siloxane of the micro-bead surface reacts with the alkyl amine positioned at the terminus of the address oligonucleotide, forming a bond between the address oligonucleotide and the micro-bead surface. Each linking micro-bead can contain one unique address oligonucleotide. Alternatively, each linking micro-bead can contain a plurality of identical address oligonucleotide sequences.

An array of micro-beads can be deposited from an aqueous suspension, optionally containing block copolymers having both photoreactive groups and ligand-attractive groups (e.g., ionic groups such as quaternary amines) thereon. Monodisperse micro-beads (e.g., between about one-half micron and 5 microns in diameter), previously loaded with address oligonucleotides, are applied to the master array support surface from suspension in an aqueous solution of the copolymers. The cationic reactive group serves to associate with the oligonucleotide sequences on the beads, while the aromatic block (e.g., oligostyrene) serves to associate with and couple to the photoreactive surface. In a particularly preferred embodiment, the linking micro-beads are provided in slight excess.

The smooth flat photoreactive master array support surface is exposed to a slurry of linking micro-beads with address oligonucleotide sequences immobilized thereon, in sufficient number to provide at least a monolayer of linking micro-beads on the master array support surface. This slurry is comprised of approximately equal numbers of beads with each of the desired address oligonucleotide sequences, plus a multiple (e.g., at least 3-fold) of beads containing no oligonucleotides. The number of oligonucleotide-coated beads is sufficient to provide a small redundancy (e.g., at least 3-fold) of each target nucleic acid-specific site.

The master array is incubated in the solution containing linking micro-beads in the dark or dim light, so as to avoid premature activation of the photogroups. As the solvent (preferably aqueous) evaporates from the slurry pool on the photoreactive surface, the uniform-sized beads are packed into a two-dimensional “crystalline” arrangement, optionally being spaced apart and positioned, e.g., using a micro-screen as described herein.

This ordered arrangement can then be “fixed” in place, for example by illumination in the dry state to form carbon-carbon bonds between the photoreactive groups on the master array support surface and the organic groups (mostly oligonucleotides) on the linking micro-beads. A patterned array of beads, having settled by gravity onto the photoreactive surface, can be photochemically fixed to the surface by illumination with long-wave ultraviolet or visible light. This illumination activates the photoreactive organosiloxane reagent, which in turn, forms bonds with the address oligonucleotides. Optionally, once the arrayed linking micro-beads have been photocoupled to the master array support surface, the unbound block copolymer can be rinsed from the master array. This rinsing removes subsequent interference (e.g., during hybridization steps) with the oligonucleotide sequences located around, but not immobilized on, the beads.

Although the use of any form of linkers is not required in the present invention, the use of micro-beads in the invention provides a combination of advantages, such as increased density of address oligonucleotide immobilized on the surface of the master array, ordered spacing of the address oligonucleotides to form discrete locations of addresses on the surface of the master array, and increased density of address oligonucleotide deposition at each discrete location on the surface of the master array (when more than one address oligonucleotide is provided in connection with each linker).

The typical master array of the present invention includes a large number of address ligands immobilized on its surface, each in a discrete location or address. The word “address,” as used in this sense, refers to a spatially-defined location upon the support surface that is sufficiently distinct to permit a ligand (e.g., oligonucleotide) bound thereto to be identified and distinguished. The result of this immobilized crystalline array provides a master array containing a large number of unique “addresses” on its surface.

A master array support surface of this sort is preferably provided in the form of a planar, non-porous solid support having at least a first surface. A plurality of different address oligonucleotides can be attached to the first surface of the solid support at a density exceeding 100 different oligonucleotides/cm², and preferably exceeding 1000 different oligonucleotides/cm², wherein each of the different oligonucleotides is attached to the surface of the solid support in a different predefined region, has a different determinable sequence, and is at least four (4) nucleotides in length, and preferably at least ten (10) nucleotides, and more preferably thirty (30) nucleotides in length.

In a preferred embodiment, the master array includes at least 1,000, and more preferably at least 10,000, different address oligonucleotides attached to the first surface of the solid support, each located in a predefined region physically separated from other regions. Given the preference for multiple sites of each oligonucleotide, the total number of oligonucleotide sites in a single array can often exceed 10,000.

In a preferred embodiment of the invention, the master array support surface contains redundant address oligonucleotides immobilized thereon, such that a particular address appears at a plurality of spatially-defined locations on the master array. As a result of this redundancy, a particular multi-ligand conjugate containing the complementary sequence to a specific address oligonucleotide will be able to bind at any of a plurality of locations on the master array. This redundancy of any particular address on the master array serves to increase the sensitivity and accuracy of the assay for detection of corresponding target nucleic acid within a sample. For example, in one embodiment, the master array will contain an address sequence that is immobilized to the support surface at between 1 and 100, preferably between 2 and 10, different locations. In such a preferred embodiment, detection of the target nucleic acid associated with the redundant address will take place at a number of different locations on the master array.

Once the address oligonucleotides have been immobilized on the surface of the master array, the spatially-defined location of each immobilized address oligonucleotide can then be determined. Suitable methods of determining the location of each address oligonucleotide include fluorescent hybridization involving the sequential addition of fluorescent-labeled, complementary sequences to the array, followed by rinsing and scanning between each addition. Fluorescent-labeled oligonucleotides complementary to each of the address oligonucleotide sequences are available or can be synthesized to contain one of several (e.g., ten or more) different fluorescent compounds commonly known to be distinguishable from each other. A plurality (e.g., ten) different oligonucleotides can be prepared, each having a different, known sequence complementary to an address sequence on the surface and each having a corresponding distinguishable fluorophore. The labeled probes can be simultaneously applied to the surface and hybridized, e.g., at 55° C. for 30 minutes. The surface is then rinsed and scanned to identify the locations of each of the ten sequences. Another set often sequences is then hybridized to the surface and scanned to identify the second set of ten sequences. This process is continued until all of the addresses are identified, after which the master array is stripped of hybridized oligonucleotides, e.g., by immersing in boiling water for two minutes, and used for preparing replicate assay arrays. One of skill in the art would readily recognize that other detection methods can be used as well.

Preferably, address oligonucleotides of the present invention include one or more pendent latent reactive (preferably photoreactive) groups covalently attached, directly or indirectly, thereto. Photoreactive groups are defined herein, and preferred groups are sufficiently stable to be stored under conditions in which they retain such properties. See, e.g., U.S. Pat. No. 5,002,582, the disclosure of which is incorporated herein by reference. Latent reactive groups can be chosen that are responsive to various portions of the electromagnetic spectrum, with those responsive to ultraviolet and visible portions of the spectrum (referred to herein as “photoreactive”) being particularly preferred.

Photoreactive groups respond to specific applied external stimuli to undergo active specie generation with resultant covalent bonding to an adjacent chemical structure, e.g., as provided by the same or a different molecule. Photoreactive groups are those groups of atoms in a molecule that retain their covalent bonds unchanged under conditions of storage but that, upon activation by an external energy source, form covalent bonds with other molecules.

The photoreactive groups generate active species such as free radicals and particularly nitrenes, carbenes, and excited states of ketones upon absorption of electromagnetic energy. Photoreactive groups may be chosen to be responsive to various portions of the electromagnetic spectrum, and photoreactive groups that are responsive to e.g., ultraviolet and visible portions of the spectrum are preferred and may be referred to herein occasionally as “photochemical group” or “photogroup”.

Photoreactive aryl ketones are preferred, such as acetophenone, benzophenone, anthraquinone, anthrone, and anthrone-like heterocycles (i.e., heterocyclic analogs of anthrone such as those having N, O, or S in the 10-position), or their substituted (e.g., ring substituted) derivatives. Examples of preferred aryl ketones include heterocyclic derivatives of anthrone, including acridone, xanthone, and thioxanthone, and their ring substituted derivatives. Particularly preferred are thioxanthone, and its derivatives, having excitation wavelength greater than about 360 nm.

The functional groups of such ketones are preferred since they are readily capable of undergoing the activation/inactivation/reactivation cycle described herein. Benzophenone is a particularly preferred photoreactive moiety, since it is capable of photochemical excitation with the initial formation of an excited singlet state that undergoes intersystem crossing to the triplet state. The excited triplet state can insert into carbon-hydrogen bonds by abstraction of a hydrogen atom (from a support surface, for example), thus creating a radical pair. Subsequent collapse of the radical pair leads to formation of a new carbon-carbon bond. If a reactive bond (e.g., carbon-hydrogen) is not available for bonding, the ultraviolet light-induced excitation of the benzophenone group is reversible and the molecule returns to ground state energy level upon removal of the energy source. Photoactivatible aryl ketones such as benzophenone and acetophenone are of particular importance inasmuch as these groups are subject to multiple reactivation in water and hence provide increased coating efficiency.

The azides constitute a preferred class of photoreactive groups and include arylazides (C₆R₅N₃) such as phenyl azide and particularly 4-fluoro-3-nitrophenyl azide, acyl azides (—CO—N₃) such as benzoyl azide and p-methylbenzoyl azide, azido formates (—O—CO—N₃) such as ethyl azidoformate, phenyl azidoformate, sulfonyl azides (—SO₂—N₃) such as benzenesulfonyl azide, and phosphoryl azides (RO)₂PON₃ such as diphenyl phosphoryl azide and diethyl phosphoryl azide. Diazo compounds constitute another class of photoreactive groups and include diazoalkanes (—CHN₂) such as diazomethane and diphenyldiazomethane, diazoketones (—CO—CHN₂) such as diazoacetophenone and 1-trifluoromethyl- 1 -diazo-2-pentanone, diazoacetates (—O—CO—CHN₂) such as t-butyl diazoacetate and phenyl diazoacetate, and beta-keto-alpha-diazoacetates (—CO—CN₂—CO—O—) such as t-butyl alpha diazoacetoacetate. Other photoreactive groups include the diazirines (—CHN₂) such as 3-trifluoromethyl-3-phenyldiazirine, and ketenes (—CH═C═O) such as ketene and diphenylketene.

Upon activation of the photoreactive groups, the reagent molecules are covalently bound to each other and/or to the material surface by covalent bonds through residues of the photoreactive groups. Exemplary photoreactive groups, and their residues upon activation, are shown as follows.

| Photoreactive | Group |
|---|---|
| aryl azides | amine |
| acyl azides | amide |
| azidoformates | carbamate |
| sulfonyl azides | sulfonamide |
| phosphoryl azides | phosphoramide |
| diazoalkanes | new C—C bond |
| diazoketones | new C—C bond and ketone |
| diazoacetates | new C—C bond and ester |
| beta-keto-alpha-diazoacetates | new C—C bond and beta-ketoester |
| aliphatic azo | new C—C bond |
| diazirines | new C—C bond |
| ketenes | new C—C bond |
| photoactivated ketones | new C—C bond and alcohol |

The photoactivatable nucleic acids of the invention can be applied to any surface having carbon-hydrogen bonds, with which the photoreactive groups can react to immobilize the nucleic acids to surfaces. Examples of appropriate substrates include, but are not limited to, polypropylene, polystyrene, poly(vinyl chloride), polycarbonate, poly(methyl methacrylate), parylene and any of the numerous organosilanes used to pretreat glass or other inorganic surfaces. The photoactivatable nucleic acids can be printed onto surfaces in arrays, then photoactivated by uniform illumination to immobilize them to the surface in specific patterns. They can also be sequentially applied uniformly to the surface, then photoactivated by illumination through a series of masks to immobilize specific sequences in specific regions. Thus, multiple sequential applications of specific photoderivatized nucleic acids with multiple illuminations through different masks and careful washing to remove uncoupled photo-nucleic acids after each photocoupling step can be used to prepare arrays of immobilized nucleic acids. The photoactivatable nucleic acids can also be uniformly immobilized onto surfaces by application and photoimmobilization.

Masks are known in the art and can be provided in the form of a transparent support material selectively coated with a layer of opaque material. Generally, masks are spatially distributed barrier materials that are applied to a substrate of interest to block application of photons from a portion of the substrate surface overlaid by the barrier material. Such masks shield the underlying portion of substrate from contact with activating light. Suitable masks are discussed, for example, in U.S. Pat. No. 5,831,070 (Pease et al., Nov. 3, 1998). Portions of the opaque material can be removed, leaving opaque material in the precise pattern desired on the substrate surface. The mask is brought into close proximity with, imaged on, or brought directly into contact with the master array support surface. “Openings” in the mask correspond to areas on the substrate where it is desirable to attach a desired moiety, such as mucleic acid, micro-beads, and the like. In some embodiments, the mask can be repositioned for multiple applications. Alternatively, multiple masks may be used.

Multi-Ligand Conjugate

The invention further includes a multi-ligand conjugate containing a plurality of active (e.g., binding or polymerizable) domains. The multi-ligand conjugate comprises a core having attached thereto a first ligand binding domain, a second ligand binding domain, and a third ligand. The first and second ligand binding domains are preferably nucleic acids, while the third ligand is preferably a moiety other than a nucleic acid. Preferably, the third ligand comprises a binding ligand or a polymerizable group. Alternatively, the multi-ligand conjugate comprises a core having attached thereto a second ligand binding domain, and a third ligand. In this embodiment, the second ligand binding domain is complementary to both the address ligand and a target ligand to be detected in a sample. The multi-ligand conjugate provides a conjugate that is capable of using the template established by the master array, to produce one or more replicate assay arrays as described herein.

The core of the multi-ligand conjugate provides an area for attachment of the first and second ligand binding domains, as well as the third ligand. The core comprises a suspendable particle or a soluble molecule, as described herein. Suitable suspended particles include micro-beads. Alternatively, the core comprises a molecular moiety, that is preferably soluble. As used herein, when the core of the multi-ligand conjugate is provided in the form of a micro-bead, the micro-bead will be referred to as a “transfer micro-bead.”

In one embodiment, the core is provided in the form of a transfer micro-bead. Micro-beads useful in preparing a multi-ligand conjugate of the invention can be of the same variety as those described above with respect to the “linking micro-beads.” Preferred beads are desirably spherical, homogeneous in size, with a specific gravity greater than that of water. Also, preferred beads have a chemical composition, or at least surface, that is subject to bonding with organic reagents. For example, homogeneous silica spheres of between about 0.5 micron and about 20 microns, preferably between about 1 micron and about 10 microns, more preferably between about 1 micron and about 5 microns (μ) diameter can be reacted with an organosiloxane reagent containing a thermochemically reactive group (e.g., epoxy), in order to couple the reagent to corresponding groups of a binding ligand.

An appropriate transfer micro-bead for use in the invention includes any three dimensional structure that is capable of being bound to a first ligand binding domain, second ligand binding domain, and third ligand, as herein described. Preferably, the transfer micro-bead has a density that is greater than water, to allow the beads to be brought into proximity with the surface of the master array for hybridization between the address oligonucleotide and the first binding domain of the multi-ligand conjugate. The transfer micro-bead can be provided in any suitable size, and nano-beads as well as micro-beads are contemplated in the invention.

As contemplated in the invention, the bead can be fabricated from virtually any insoluble or solid material. For example, the bead can be fabricated from silica gel, glass, nylon, Wang resin, Merrifield resin, Sephadex, Sepharose, cellulose, magnetic beads, Dynabeads, a metal surface (e.g. steel, gold, silver, aluminum, silicon and copper), a plastic material (e.g., polyethylene, polypropylene, polyamide, polyester, polyvinylidenedifluoride (PVDF)) and the like, and combinations thereof. Examples of suitable micro-beads are described, for example, in U.S. Pat. No. 5,900,481 (Lough et al, issued May 4, 1999). Other examples of suitable micro-beads are available, for example, from Interfacial Dynamics Corporation (Portland, Oreg.).

In another embodiment, the core is provided in the form of a molecular core. Preferably, the molecular core is soluble. Suitable molecular cores include multifunctional polymers or polyfunctional monomeric molecules. Examples of multifunctional polymers include linear or branched polymers, or dendrimers. Examples of suitable polyfunctional monomeric molecules include cyanuric chloride, homocysteine, cysteine, lysine, aspartic acid, glutamic acid, serine, and the like.

In one embodiment, the multi-ligand conjugate of the invention further includes a first ligand binding domain. In a preferred embodiment, the first ligand binding domain comprises a nucleic acid, preferably an oligonucleotide. Oligonucleotides are selected for the first binding domain by preparing the complementary sequence to the random sequences comprising the address ligands of the master array. Once the sequences of the address ligands are known, the sequence of the first ligand binding domain is prepared by first determining the complementary nucleic acid sequence to the address oligonucleotide, using standard Watson-Crick base pairing rules, and then synthesizing the determined nucleic acid sequence. This preparation can be accomplished using any suitable methods known in the art, for example, by solid phase DNA synthesis. Once the oligonucleotides for the first binding domain have been synthesized, a terminus of each oligonucleotide can be modified to contain an alkylamine group on a spacer, when desired.

The length of the oligonucleotides is optimized for desired hybridization strength and kinetics. Usually, the length of the first ligand binding domain is in the 20-50 nucleotide range. Preferably, the length of the first ligand binding domain is selected based upon the length of the address ligand on the master array. In a preferred embodiment, the sequences of the first binding domain are not complementary either to one another or to any known natural gene sequence and/or gene fragment with significant probability of being present in the sample to be tested. As a result, the first binding domain oligonucleotide sequences will hybridize only with their respective complementary address oligonucleotide sequences immobilized on the master array support surface. This avoids cross-reactivity (for example, cross-hybridization) between the first ligand binding domain, which is responsible for placing the multi-ligand conjugate at a spatially-defined location on the assay array support surface, and the target nucleic acid sequences screened in the test sample.

In a preferred embodiment, the second ligand binding domain of the multi-ligand conjugate also comprises a nucleic acid, preferably an oligonucleotide. Oligonucleotides are selected for the second binding domain by first selecting the target ligands (e.g., genes and/or gene fragments) to be detected by the assay array to be fabricated in accordance with the invention. The sequences of these genes and/or gene fragments to be detected may be known, or may be determined using techniques well known in the art (e.g., Sanger dideoxy method, PCR sequencing, etc.). Once the sequences of the target nucleic acid is known, the sequence of the second ligand binding domain is prepared by first determining the complementary nucleic acid sequence to the target nucleic acid, using standard Watson-Crick base pairing rules, and then synthesizing the determined nucleic acid sequence. This preparation can be accomplished using any suitable methods known in the art, for example, by solid phase DNA synthesis. Once the second binding domains have been chosen, a terminus of each oligonucleotide can be modified to contain an alkylamine group on a spacer, when desired.

Similar to the first ligand binding domain, the lengths of the oligonucleotides for the second ligand binding domains are optimized for desired hybridization strength and kinetics, preferably in the 10-100 nucleotide range, more preferably in the 20-50 nucleotide range. Preferably, the second binding domain sequences are selected to provide sufficient specificity for particular target ligands in a sample, while avoiding cross-reactivity, such as cross-hybridization, with either the address ligands on the master array, or the first ligand binding domain of the multi-ligand conjugate.

In an alternative embodiment, the second ligand binding domain is selected to be complementary to an address ligand of the master array, as well as a target ligand to be detected in a sample. In this embodiment, the sequence of the second ligand binding domain is determined by selecting the target ligands (e.g., genes and/or gene fragments) to be detected by the assay array to be fabricated in accordance with the invention. The sequence of the second ligand binding domain is prepared by determining the complementary nucleic acid sequence to the target nucleic acid, using Watson-Crick base pairing rules, and then synthesizing the determined nucleic acid sequence, as discussed above. The address ligands in this embodiment will then be fabricated to include sequence that is substantially complementary to the target ligand. According to this embodiment, there is no need to provide the multi-ligand conjugate with a first ligand binding domain, since the second ligand binding domain will hybridize with the address ligands of the master array.

The multi-ligand conjugates can be fabricated to include oligonucleotides that are complementary to, and thus will hybridize with, any desired target nucleic acid found in a biological sample, and which has a known, or determinable, sequence. In one embodiment, the various second binding domains of the multi-ligand conjugates of the present invention can comprise a wide array of oligonucleotides that are complementary to various target nucleic acids found in a biological sample.

In an alternative embodiment, the second binding domains of the multi-ligand conjugates will be fabricated such that the resulting assay array will locate target nucleic acids of a particular family of naturally-occurring genes. One example of this particular embodiment would be a set of multi-ligand conjugates containing second binding domains complementary to the 50-60 known target sequences of cystic fibrosis. Thus, the resulting assay array would be fabricated such that it detects the presence of the sequences known or suspected to be associated with this disorder only.

The third ligand of the multi-ligand conjugate is a molecule that transfers the multi-ligand conjugate to the assay array prepared according to the invention. In one embodiment, once the multi-ligand conjugates have hybridized to the master array at the desired address locations, the third ligand allows the multi-ligand conjugate to be transferred to the assay array while maintaining the positional arrangement of nucleic acids established by the master array. Preferably, the third ligand is provided as a binding ligand or a polymerizable group.

In one embodiment, the third ligand is a binding ligand. As used herein, “binding ligand” means any ligand that is capable of binding to a binding partner. Preferably, the binding ligand is a member of a specific binding pair that is capable of binding with a complementary binding moiety. As contemplated in the invention, the binding ligand is a biological or synthetic molecule that has high affinity for another molecule or macromolecule, through covalent or non-covalent bonding. Preferably, the binding ligand contains (either by nature or by modification) a functional chemical group (such as a primary amine, sulfhydryl group, aldehyde, or the like), an epitope (antibody), a hapten or a ligand, that allows it to covalently react or non-covalently bind to a common functional group. For example, when the third ligand comprises avidin or streptavidin, the multi-ligand conjugate can be bound to a surface coated with biotin or derivatives of biotin such as imino-biotin. It will be appreciated that the binding members can be reversed, e.g., a biotin-coated bead can bind to a streptavidin-coated solid support. Other specific binding pairs contemplated for use in the invention include haptens (such as digoxigen, 2,4,-dichlorophenoxyacetic acid, trinitrophenyl) and high-affinity antibodies, hormone-receptor, enzyme-inhibitor, antibody-antigen and the like.

In one embodiment, the third ligand is provided in the form of a biotin-poly(alkyleneoxide) amine derivative. Such a derivative can be prepared, for instance, by reacting an N-oxysuccinimide ester of biotin with a poly(ethyleneoxide) diamine of approximately the same molecular length as the oligonucleotide derivatives to be placed on the same core or micro-bead.

The third ligand can be attached directly to the core or transfer micro-bead of the multi-ligand conjugate, or indirectly by the use of a spacer, in a manner that allows the ligand to be free of steric hindrance (e.g., steric hindrance caused by proximity of the ligand to the transfer micro-bead). In other words, use of a spacer to attach the third ligand will ensure that the ligand is sufficiently distant from the core or surface of the transfer micro-bead to allow it to perform its desired function, e.g., polymerize or bind with a corresponding binding partner contained on the assay array surface.

As used herein, a “spacer” for attachment of the third ligand to the core is a molecule that allows attachment of the third ligand to the core in a spatial relationship that does not interfere with the function or reactivity of the third ligand. Suitable spacers include polyethylene glycol, as well as water-soluble bi- or poly-functional monomers such as acrylamides, vinylpyrrolidones, carbohydrates, and the like. Preferably, such water-soluble bi- or poly-functional monomers comprise oligomers or polymers. In a preferred embodiment, the spacer comprises a hydrophilic spacer, for example, a polyethylene glycol spacer, of sufficient length to allow the binding domain to be free of steric hindrance.

In one embodiment, the multi-ligand conjugate of the present invention further comprises a polymerizable group. As used herein, “polymerizable group” refers to a molecule that is capable, under suitable conditions, of undergoing polymerization, a chemical reaction in which relatively simple molecules combine to form a macromolecule or a chain-like molecule. The third ligand of the multi-ligand conjugate can be provided in the form of a polymerizable group, e.g., to allow the conjugates to form into a stable film while in position on the master array.

Examples of suitable polymerizable groups include free-radical generators such as vinyl, acrylic, maleic, itaconic, unsaturated fatty acids, and other ethylenically- and acetylenically-unsaturated hydrocarbon groups. In a preferred embodiment, the polymerizable groups are adapted to undergo addition polymerization, in the presence of added monomers and other reagents (e.g., initiators), to form a stable film. Polymerization can take place via any suitable reaction, including aqueous free-radical solution polymerization, exposure to heat, high energy radiation, ultrasonic waves, ultraviolet radiation, and ionic polymerization catalysts to produce water-soluble or swellable polymers. Further, polymerization of the groups can take place via base-catalyzed hydrogen-transfer polymerization. Those skilled in the art, given the present description, will appreciate the manner in which such groups can be used to copolymerize the ligands with other monomers, and in turn their corresponding conjugates, into the form of an integral polymeric structure, e.g., a film backing, which can in turn be used as or transferred to an assay array support surface.

In this embodiment, once the address oligonucleotides of the master array have each hybridized with their respective first binding domains of the multi-ligand conjugates, the polymerizable group can be reacted, using reagents and conditions within the skill of those in the art, to allow the formation of a polymer film. The polymerizable groups are copolymerized into the film backing while substantially maintaining their respective positions determined by the master array. The film can be used as is, or optionally stabilized (e.g., gelled, laminated with another layer, or otherwise solidified) such that it is sufficiently stable (e.g., self supporting) to maintain the spatially-defined locations of each “address.” Simultaneously or thereafter, this polymeric backing can be transferred to, or otherwise incorporated into, an assay array surface in a manner that maintains the desired orientation of conjugates.

In accordance with the invention, the individual ligands of the multi-ligand conjugate can be attached to a core atom or molecule in any suitable manner and/or order, e.g., individually or together (e.g., in linear sequence and at a single location or at a plurality of locations). For example, in one embodiment, the ligands are attached to the core simultaneously, e.g., under similar reaction conditions. Optionally, the ligand serving as the third ligand is attached to the core after hybridization between the address oligonucleotide of the master array and the complementary oligonucleotide provided by the multi-ligand conjugate. In this embodiment, the third ligand is present as a separate reagent; however, attachment of the third ligand is capable of taking place under the same or similar reaction conditions as the first and second binding domains. In yet another embodiment, each of the ligands is attached to the core under separate conditions of separate reactions.

The sequence of attachment of these ligands to the core of the multi-ligand conjugate is not considered to be crucial to the present invention, and so long as the individual active (e.g., binding) domains are available for binding their specific complement, they can be attached to the core or to one another in any order. In a particularly preferred embodiment, each individual binding domain is attached to the core individually, at a plurality of locations on the surface.

In one embodiment, epoxy-glass surfaces are formed on the surface of the transfer micro-bead, which is thereafter reacted with the first and second ligand binding domains, as well as the third ligand. In this embodiment, glycidoxypropyltriethoxy siloxane is reacted with glass micro-beads under published reaction conditions (See “Grafting Rates of Amine-Functionalized Polystyrenes onto Epoxidized Silica Surfaces,” Macromolecules, 33:1105-1107 (2000)), to provide epoxy-glass surfaces. The three binding domains of the present invention, each terminated with alkylamine groups on spacers, can be readily coupled with the epoxy groups on the surface of the bead.

First and second oligonucleotide binding domains can be attached to the transfer micro-bead in any suitable fashion, e.g., alkylamine-terminated nucleic acids can be synthesized and thermochemically attached. As described above, the first binding domain comprises an oligonucleotide that is complementary to the address oligonucleotide immobilized onto the master array support surface, while the second binding domain is complementary to a chosen target nucleic acid suspected or known to be in a sample to be tested. As discussed above, the sequences of the first ligand binding domain are preferably not complementary either to one another or to any known natural gene sequence and/or gene fragment with significant probability of being present in the biological sample.

In one embodiment, the first and second ligand binding domains are provided in the form of alkylamine nucleic acid derivatives, and the third ligand is provided in the form of a biotin hydrazide. The core is provided as a micro-bead that is provided with a photoreactive poly-N-oxysuccinimide (polyNOS) or epoxide surface. The alkylamine derivatives and biotin hydrazide react with the reactive surface of the core to allow attachment of the binding ligands and third ligand. Alternatively, 5′-OH oligonucleotide and allylamine oligonucleotide derivatives are used as the first and second ligand binding domains, and the third ligand is provided as biotin hydrazide (binding ligand) or allylamine (polymerizable group, for in situ formation of patterned film), with triazine trichloride as a soluble molecule core.

In an alternative embodiment, the third ligand is provided in solution, rather than attached to the core of the multi-ligand conjugate. In this embodiment, the third ligand is incorporated after hybridization of the master array address oligonucleotide with its complementary first binding domain of the multi-ligand conjugate. Therefore, in this embodiment, the third ligand is incorporated into the complex formed on the master array after attachment of the multi-ligand conjugate, and before the assay array is brought into binding proximity with the master array. Preferably, the addition of the third ligand does not require different reaction conditions from that of the first and second binding domains; rather, the addition of the third ligand can take place under the same conditions.

In a preferred embodiment, the third ligand is attached to the multi-ligand conjugate using photoreactive chemistry. In this embodiment, the surface of a micro-bead is pretreated, for example, with an organosiloxane in order to prepare the surface for reacting with photoreactive compounds. In this embodiment, the third ligand is attached to a hydrophilic spacer, which is, in turn, attached to a photoreactive group. The photoreactive group allows bond formation between the spacer (attached to the binding ligand) and the core of the multi-ligand conjugate.

The third ligand is preferably bound covalently to the core, whether the core comprises a suspendable particle or a soluble molecule. In one embodiment, the third ligand derivative includes a reactive group selected to form a covalent bond with a target group found or placed on the core of the multi-ligand conjugate. For example, the core may contain epoxy or active ester groups and the third ligand may contain amine or hydrazide groups; these two classes of reactive groups will react to form covalent bonds when placed together in aqueous, slightly alkaline conditions. When the core comprises a suspendable particle, for example, the biotin-poly(alkyleneoxide) amine derivative would be reacted with a soluble copolymer containing photoreactive groups and N-oxysuccinimide (NOS) ester groups (as would amine-containing derivatives of the other two ligands or ligand-binding domains). This multi-ligand conjugate derivative would be photochemically bound to the core particle.

In an alternative embodiment, wherein the invention includes the use of porous linking micro-beads immobilized onto the surface of the master array, and covalently bound to address oligonucleotides, the transfer micro-bead as the core of the multi-ligand conjugate is not required. In this embodiment, the porous linking micro-beads can be immobilized onto the surface of the master array, each porous micro-bead having a plurality of address oligonucleotides attached to its surface. According to this embodiment, the multi-ligand conjugates comprise a first binding domain complementary to the address oligonucleotide, a second binding domain complementary to a target ligand found in a biological sample, and a third ligand comprising a binding ligand. The multi-ligand conjugate preferably does not contain a transfer micro-bead, but rather a chemical (e.g., molecular) core.

A plurality of multi-ligand conjugates are applied to the master array and incubated under conditions suitable to allow hybridization of the address sequence of the master array with its complementary oligonucleotide contained within the multi-ligand conjugate. Those skilled in the art will be able to determine suitable conditions of temperature, salt concentration, and buffer composition for hybridization of address oligonucleotides and first binding domains. For a discussion of hybridization conditions and methods for applying them, see pages 3-15, 62-66, 73-112, Nucleic Acid Hybridization: A Practical Approach, Hames and Higgins, eds. (1985), the disclosure of which is incorporated herein by reference.

Assay Array Support

The assay array support of the invention comprises a support surface that is preferably coated with binding sites for the third ligand of the multi-ligand conjugate. The assay array support provides the support for a replicate array to be fabricated using the master array.

The assay array support can be fabricated from any suitable material to provide an optimal combination of such properties as stability of dimension, shape, and smoothness. Suitable materials for use in fabricating the assay array include those described as being useful for the master array itself, and preferably include silicon and glass. In a particularly preferred embodiment, the assay support is provided with planar dimensions of between about 0.5 cm and about 7.5 cm in length, and between about 0.5 cm and about 7.5 cm, preferably between about 1 cm and about 2 cm, in width. Preferably, the assay array support is of comparable dimensions to the master array, in order to provide direct spatial relationships.

In yet another embodiment, multiple arrays can be replicated onto a single assay array support. In this embodiment, one or more master arrays are used to create individual replicate arrays in defined areas on the assay array support. The resulting assay array contains multiple probe arrays on a single support surface, and the composition of each array can be tailored for a particular use, as desired.

As used herein, a “binding site” refers to an area on the surface of the assay array that is capable of binding the third ligand of the multi-ligand conjugate. Preferably, the binding sites comprise members of a specific binding pairs that are capable of binding with a complementary binding moiety, e.g., the third ligand of the multi-ligand conjugate, in a covalent or non-covalent manner. Suitable specific binding pairs are described herein with respect to the third ligand of the multi-ligand conjugate. One of skill in the art can select suitable binding sites based upon the composition of the multi-ligand conjugate and desired use of the assay array.

The surface of a discrete assay array support can be pretreated to facilitate attachment of the oriented conjugate layer. In a preferred embodiment, the assay array is provided in the form of a silicon chip that has been cleaned, polished and treated to provide a smooth silicon oxide surface. The silicon oxide surface can then be treated with an organosiloxane reagent (as described herein) to provide a smooth photoactivatable surface.

In one embodiment, the assay array support of the present invention can be coated with binding partner molecules available to form bonds with the third ligand of the multi-ligand conjugate. The assay array support surface can be prepared by coating the surface with a photoactivatable compound (e.g., photoreactive siloxane reagent) to render the surface photoreactive. Thereafter, the assay array support surface can be exposed to a solution containing binding partner molecules (e.g., avidin), and the solution illuminated, in order to allow bond formation between the binding partner and surface of the assay array. Optionally, the surface of the assay array is passivated prior to and/or after exposure to the binding partner, e.g., with a surfactant (e.g., a biotin triblock copolymer), to prevent non-specific binding of molecules found in the test sample to the assay array support surface.

In a preferred embodiment, the binding partner is provided in the form of a monolayer upon the assay array support surface. Optionally, the binding partner can be provided in the form of a three-dimensional layer, e.g., an avidin-hydrogel composite of sufficient thickness, for example, up to about 20 microns (μ) thickness. Thickness of the three-dimensional layer will depend upon such factors as the molecular weight of the polymer used, the swellability of the polymer (e.g., hydrophilicity), and the quantity (e.g., concentration and volume) of material applied. Suitable thickness is about 0.1μ to about 20μ, preferably about 0.5μ to about 10μ, more preferably about 0.5μ to about 5μ. Such a structure can be obtained, for instance, by loading the photoreactive assay array surface with a solution of avidin plus photoreactive hydrogel (e.g., copolymer of benzoylbenzamidopropyl methacrylamide with vinylpyrollidone or acrylamide) and illuminating while wet.

In another preferred embodiment, the linking partner is provided by a thin film of photopolymer that is applied to a dimensionally stable assay array support surface. A self-assembling monolayer film of passivating surfactant containing a high surface density (e.g., 0.1 pmol/mm²) of high-affinity ligand group (e.g., biotin) is applied to the photoreactive surface. The monolayer surfactant film is then photocoupled to the surface, and any unbound surfactant is rinsed away. The passivated, ligand-loaded surface is saturated with high-affinity binding partner molecule (e.g., x-avidin), and any excess binding partner is rinsed away. Suitable high-affinity binding partner molecules include, for example, x-avidin film, or high-affinity antibody (e.g., anti-digoxygenin).

Optionally, the system further includes other components as well, including for instance, reagents or other mechanisms for use in disassociating complementary, hybridized oligonucleotides (e.g., the address oligonucleotide of the master array and its complementary oligonucleotide carried by the multi-ligand conjugate). For example, suitable mechanisms for disassociating the hybridized DNA include techniques or reagents that alter the temperature, pH, or salt concentration of the system. One of skill in the art could apply such mechanisms to dissociate hybridized DNA and achieve the invention herein.

Optionally, the system further comprises a scanner, as commercially available (e.g., a confocal fluorescence microscope as available from Molecular Dynamics) to detect the hybridization of nucleic acid targets to specific addresses.

Method

In another aspect, the invention provides a method for reproducibly preparing a nucleic acid array, the method comprising:

a) providing a master array having a support surface;

b) immobilizing a plurality of address ligands on the master array support surface in the form of a patterned array;

c) determining the pattern of the immobilized address oligonucleotides;

d) providing a plurality of multi-ligand conjugates, each multi-ligand conjugate comprising: (i) a core; (ii) at least one molecule of a first ligand binding domain, comprising a ligand selected to bind in a complementary manner to an address ligand of the master array, (iii) at least one molecule of a second ligand binding domain, comprising a ligand selected to bind in a complementary manner to a particular target ligand, and (iv) at least one molecule of a third ligand, wherein the first ligand binding domain, second ligand binding domain, and third ligand are attached to the core;

e) contacting and incubating the multi-ligand conjugates with the master array support surface under conditions suitable to allow each address ligand to bind its complementary first ligand binding domain of the multi-ligand conjugates;

f) providing an assay array support surface,

g) contacting the bound multi-ligand conjugates with the assay support surface under conditions suitable to permit the conjugates to be transferred to the assay support in a pattern corresponding to their pattern on the master array; and

h) disassociating the first ligand binding domain from the master array support in a manner that permits the conjugates to remain upon the assay support surface in the desired pattern.

In another aspect, the invention provides a method for reproducibly preparing a nucleic acid array comprising the steps of:

a) providing a master array having a support surface;

b) immobilizing a plurality of address ligands on the master array support surface in the form of a patterned array;

c) determining the pattern of the immobilized address oligonucleotides;

d) providing a plurality of multi-ligand conjugates, each multi-ligand conjugate comprising: (i) a core; (ii) at least one molecule of a second ligand binding domain, comprising a ligand selected to bind in a complementary manner to a particular address ligand and to a particular target ligand, and (iii) at least one molecule of a third ligand, wherein the second ligand binding domain and the third ligand are attached to the core;

e) contacting and incubating the multi-ligand conjugates with the master array support surface under conditions suitable to allow each address ligand to bind its complementary second ligand binding domain of the multi-ligand conjugates;

f) providing an assay array support surface,

g) contacting the bound multi-ligand conjugates with the assay support surface under conditions suitable to permit the conjugates to be transferred to the assay support in a pattern corresponding to their pattern on the master array; and

h) disassociating the second ligand binding domain from the master array support in a manner that permits the conjugates to remain upon the assay support surface in the desired pattern.

Optionally, the method further comprises the step of using the assay array support surface fabricated in step h) to prepare a second assay array. A second set of multi-ligand conjugates are used to generate a second assay array that is a substantially identical copy of the original master array. The second set of multi-ligand conjugates can be fabricated according to the teachings herein. In this embodiment, the nucleic acid sequences of the second assay array are substantially identical to the nucleic acid sequences of the original master array.

In a preferred embodiment, immobilization of the address ligands onto the master array support surface is random, and the location of each address is determined after the address ligands are immobilized on the surface. Preferably, the address ligand, first ligand binding domain, and second ligand binding domain are nucleic acid sequences, and the third ligand comprises a binding ligand or a polymerizable group. In use, the core of the multi-ligand conjugate bearing three or more ligands can be mixed in equal numbers and suspended in aqueous buffer. This suspension can contain an excess (over the hybridization capacity of the micro-beads on the master array) of each transfer micro-bead carrying the oligonucleotide sequence complementary to a characteristic oligonucleotide sequence of a target nucleic acid expected to be detected in a sample.

The master array surface can be flooded with this suspension of the chosen mixture of multi-ligand conjugates and solid-state hybridization can be allowed to proceed to near equilibrium. The excess multi-ligand conjugates can then be recovered from the hybridized master array by rinsing, and the bound conjugates either copolymerized to form a stable backing (when the third ligand comprises a polymerizable group) or bound to corresponding binding partners (when the third ligand comprises a binding ligand) on the assay array. The master array is then available for a repeat replication process.

The master array can be used repeatedly to prepare corresponding assay arrays, which in turn can be packaged, stored and transported, e.g., in the wet or dry state. Upon removal from its package, an assay array will be ready to use in a routine hybridization assay protocol with a scanner. Moreover, the present invention provides a master array that is versatile to use—the master array can be used and reused, to create substantially identical arrays or to change the function of the array (e.g., to alter the oligonucleotide sequences complementary to a characteristic oligonucleotide sequences of target nucleic acid, such that the user can alter the target nucleic acid to be detected in a sample).

Those skilled in the art, given the present description, will appreciate the manner in which commercially available microarrays can themselves be used as master arrays of the present invention, to be replicated one or more times to form corresponding assay arrays. In this case, the individual nucleic acids of the commercial array, regardless of their original specificity, can each be considered as mere address sequences, and used in the manner described herein to provide assay arrays of any desired specificity. The user can determine the sequences of each address and fabricate the first binding ligand of the multi-ligand conjugate using the teachings herein.

In yet a further aspect, and particularly where the third ligand is provided in the form of a binding ligand, the present invention provides a sandwich array comprising:

a) a master array comprising a support surface having a plurality of address ligands immobilized thereon;

b) a plurality of multi-ligand conjugates, each multi-ligand conjugate comprising a core having attached thereto; (i) molecules of a first ligand binding domain, each molecule comprising a ligand selected to bind in a complementary manner to a particular address ligand of the master array, (ii) molecules of a second ligand binding domain, each molecule comprising ligand selected to bind in a complementary manner to a characteristic target ligand; and (iii) at least one molecule of a third ligand, wherein the first ligand binding domain is bound to the corresponding address ligand immobilized onto the master array; and

c) an assay array support comprising a support surface coated with attachment sites for the third ligand, wherein the attachment site of the assay array is bound to the corresponding binding ligand of the multi-ligand conjugates.

Optionally, the sandwich array further comprises a linking micro-bead immobilized onto the surface of the master array, the linking micro-bead having attached thereto a plurality of address oligonucleotides.

Kit

In yet another alternative embodiment, the present invention provides a corresponding kit comprising one or more components for reproducibly preparing a nucleic acid array, the kit components selected from the group consisting of:

a) a master array comprising a support surface having a plurality of “address” ligands immobilized thereon, the ligands being immobilized in the form of a patterned array;

b) a plurality of multi-ligand conjugates, each multi-ligand conjugate comprising a core having attached thereto: (i) molecules of a first ligand binding domain, each molecule comprising a ligand selected to bind in a complementary manner to a particular address ligand of the master array, (ii) molecules of a second ligand binding domain, each molecule comprising a ligand selected to bind in a complementary manner to a characteristic target ligand, and (iii) at least one molecule of a third ligand;

c) an assay array support comprising a support surface for the replicate array coated with attachment sites for the third ligand.

Optionally, the kit of the invention further comprises reagents for performing the assay described herein, such as suitable buffers to provide appropriate pH levels, salt concentrations, and the like to perform hybridization. The kit can further comprise an array reader for determining the location of address oligonucleotides on the master array and the presence of hybridized target nucleic acids on the assay array.

Reusable Nucleic Acid Array

In yet another alternative embodiment, the present invention provides a method and system for directly preparing a replicable array in the form of a reusable nucleic acid array. In this embodiment, the surface to which the nucleic acid is immobilized is provided by an optical fiber array, the master array support surface being provided by the distal ends of the fibers themselves. Optionally, and preferably, micro-wells are etched into the distal ends of the optical fibers.

In such an embodiment, a conventional optical fiber bundle can be adapted for use as a specific binding array biosensor system for detecting a variety of target nucleic acids in a sample. Commercial sources provide optical imaging fiber bundles comprising thousands (e.g., 3,000-100,000) of hexagonally packed, individually-coated optical fibers. Each fiber in the array carries its own, isolated optical signal from one end of the fiber to the other. Individual specific binding ligands can be bound to one end of each fiber and the other end is functionally connected to a optical signal analyzer (e.g., a CCD camera).

The assay end (containing specific-binding ligands) of the fiber bundle is exposed to a sample solution containing the unknown target and other necessary reagents (if any) of the optical assay system. The optical signal pattern is recorded and analyzed at the other end of the bundle, using the image analyzer system.

Applicant has found that oligonucleotide binding domains, complementary to target sequences, can be attached to the optical assay array support surface in any suitable manner, e.g., either directly or by means of reversibly or irreversibly bound linking molecules or micro-beads. In turn, the ultimate location of the oligonucleotide binding domains can be determined (e.g., by optical means and assay), preferably controlled (e.g., by the use of reusable address domains), and optionally replicated (e.g., by the use of reversible linkers) onto flat surface.

In one preferred embodiment, the system comprises a plurality of multi-ligand conjugates, each multi-ligand conjugate comprising molecules of a) a first oligonucleotide binding domain adapted to bind with a corresponding address ligand, and b) a second oligonucleotide binding domain complementary to a characteristic oligonucleotide sequence of a target nucleic acid (e.g., a gene and/or gene fragment). The address ligands are immobilized onto the distal end of the master array, either directly, or by the use of a linker or micro-bead. The multi-ligand conjugates would then load each of the addressed fibers with a known analyte-specific ligand. The set of analyte-specific ligands on the fibers in the bundle can be changed at will.

In another embodiment, attachment of the oligonucleotide binding domain is accomplished using a micro-bead. In this embodiment, the micro-bead is prepared such that it is attached to the oligonucleotide binding domain. The micro-bead and binding domain are then attached to the assay array support surface. Attachment of the micro-bead can be accomplished using any suitable binding pair, such as, for example, avidin-biotin. In an alternative embodiment, photoreactive reagents can be used to attach the micro-bead to the assay array support surface. Attachment of the address oligonucleotide to the micro-bead, as well as attachment of the micro-bead to the surface of the master array, can be accomplished using any suitable method herein described.

Optionally, the system further comprises an image analyzer. In this embodiment, the bundle of fibers is connected at one end to an optical sensor that collects signals and processes information from these multiple sensors (e.g., fibers).

In such an approach the invention provides a reusable nucleic acid array, in which the user is able to change the function of the nucleic acid array for each particular use. For example, the linker or micro-bead can be coated with iminobiotin, and the master array support surface can be coated with biotin. The linker or micro-bead can thus be reversibly attached to the assay array support surface. The linker or micro-bead can be dissociated by altering the pH such that the lower pH causes the linker or micro-bead to dissociate with the assay array support surface.

In one embodiment, micro-wells can be etched into the distal end of each optical fiber using techniques known in the art. For example, a wet chemical etching procedure can be used to selectively etch the cores of the individual fibers. This technique takes advantage of the difference in etch rates between the core and cladding materials of the fiber. By controlling the etching time, those of skill in the art will appreciate the manner in which high-density, ordered micro-well arrays of known shape and well volume are obtained. Well architecture is determined by the preformed imagining fiber. Since each micro-well is contained at the end of its own optical fiber, each well can serve as an individual sensor. Preferably, the diameter of the etched micro-wells is slightly larger than that of individual linking micro-beads used in connection therewith.

In one embodiment, for instance, a solution containing a plurality of micro-beads having one or more oligonucleotide binding domains attached thereto is added to the surface of a bundle of optical fibers. Preferably, the oligonucletode binding domains are complementary to a target nucleic acid to be detected and/or quantitated. The individual linking micro-beads randomly settle into each micro-well as the solution is allowed to evaporate from the optical fiber bundle surface. Optionally, and in the event a photoreactive compound is used in connection with this embodiment, the optical fibers are illuminated, to allow the linking micro-bead to photocouple to the surface of each optical fiber. Optionally, excess micro-beads are then washed from the surface of the fiber bundle.

For instance, immobilization of a bead or oligonucleotide binding domain onto the distal end of the fiber, within the micro-well, can be accomplished using photoactivatable compounds to form bonds with the well surface. In one embodiment, the well surface of the optical fiber is coated with a photoreactive siloxane reagent, and thereafter, a solution containing free (i.e., unbound) oligonucleotide binding domain is applied to the well surface. The solution is then illuminated, activating the photoreactive groups and causing bond formation between the oligonucleotide binding domain and well surface. Illumination is accomplished by passing activating light through the fiber into the well.

As discussed above, each individual fiber of the fiber bundle can be etched at one end to provide a concave receptacle for a micro-bead. A photoreactive organosiloxane reagent can be prepared by bonding a commercial organosiloxane reagent, such as aminopropyldimethyl methoxy siloxane, with a thermochemically reactive photoreagent, such as the acid chloride of benzoylbenzoic acid. The photoreactive siloxane reagent is reacted with the concave silicon oxide surface of the etched optical fibers in the image analyzer bundle. In an alternative embodiment, each micro-well is coated with x-avidin to create a surface that will react with biotin. In this embodiment, the micro-beads are coated with biotin or iminobiotin derivatives. Any specific binding pair, as described herein, will be suitable for such attachment.

Preferably, the linking micro-beads are spherical, and of a diameter approximately equal to (or slightly less than) that of the individual optical fibers. The linking micro-beads of the present invention are fabricated from any suitable material, including, for example, glass, polystyrene, polyvinylbenzophenone, photopolysiloxane-coated glass. Preferably, the micro-beads have a specific gravity greater than that of water, and have a chemical composition that is subject to bonding with organic reagents.

The oligonucleotide derivatives can be synthesized according to published and commercially utilized methods, as described in more detail herein. The length of the oligonucleotide sequences are optimized for desired hybridization strength and speed (usually in the 20-50 nucleotide range). The oligonucleotide sequences are complementary to the chosen target nucleic acid.

The “address oligonucleotide” derivatives can be coupled to the epoxy-activated linking micro-beads for the optical fiber ends. In a preferred embodiment, for example, homogeneous silica spheres of approximately the fiber diameter are reacted with a commercial organosiloxane reagent containing a thermochemically reactive group (e.g., epoxy) useful for coupling to amine groups of the oligonucleotide derivatives in this invention. Glycidoxypropyltriethoxy siloxane is reacted with the glass micro-beads under published reaction conditions, to provide an epoxy-glass surface. The address oligonucleotide derivatives of this invention terminate with alkylamine groups on spacers to couple readily with the epoxy groups on the surface of the linking micro-beads.

The bundle of etched photoreactive optical fibers is exposed to a slurry of linking micro-beads (in sufficient number to provide at least one type of address oligonucleotide per fiber), each micro-bead having address oligonucleotide sequences immobilized on its surface. Preferably, one oligonucleotide binding domain is bound to each optical fiber well. In this embodiment, the master array is reusable. This slurry is comprised of approximately equal numbers of beads with each of the desired address oligonucleotide sequences. The number of beads providing oligonucleotide sequences is preferably sufficient to provide a redundancy (at least 3-fold) of each target nucleic acid specific site. As the solvent (preferably aqueous) evaporates from the slurry pool on the photoreactive surface in the dark or dim light, the uniform-sized beads are left in the concave pockets at the ends of the ordered arrangement of fibers. The linking micro-beads providing oligonucleotide sequences are then “fixed” by illumination in the dry state to form carbon-carbon bonds between the photoreactive groups on the optical fiber surface and the organic groups (mostly oligonucleotides) on the address oligonucleotide beads. Excess micro-beads are rinsed from the system.

The resultant optical fiber bundle will have a stable and ordered arrangement of the oligonucleotides complementary to target nucleic acid sequences on one end of its fibers, and the locations of the oligonucleotide sequences can be then determined by conventional methods of fluorescent hybridization, comprising sequential addition of fluorescent-labeled, complementary sequences to the array, with rinsing and array reading between each addition, as discussed in detail herein.

In one preferred embodiment, complementary sequences are labeled with fluorophores. Fluorescent-labeled oligonucleotides complementary to each of the sequences are synthesized, each containing several (e.g., one of ten) different fluorescent compounds that can be distinguished from each other. Each oligonucleotide, therefore, has a known sequence and a known identifiable label. The several oligonucleotides, each having a different, known sequence complementary to an oligonucleotide sequence on the surface and one of a comparable number of distinguishable fluorophores, are simultaneously applied to the surface and hybridized at 55° C. for 30 minutes.

The surface is then rinsed and analyzed to identify the locations of each of the sequences. Another set of sequences is then hybridized to the surface and scanned to identify the second set of sequences. This process is continued until all of the locations are identified, after which the master array is stripped by immersing in boiling water for two minutes. It is then ready for use to detect the presence of target nucleic acids in a sample.

In use, the master array is exposed to a test solution containing a plurality of target nucleic acid sequences (e.g., genes and/or gene fragments). The test solution is labeled via a fluorescent label to permit visualization of the results. The test solution is incubated under conditions to allow the oligonucleotide binding domain to hybridize to the target nucleic acid contained within the sample. Unbound solution is then rinsed from the master array. The hybridized nucleic acid sequences are visualized using the CCD camera of the array system described above.

The assay arrays are washed with phosphate buffered saline containing 0.05% Tween 20 (PBS/Tween), then blocked with hybridization buffer, which consists of 4× SCC (0.6 M NaCl, 0.06 M citrate, pH 7.0), 0.1% (w/v) lauroylsarcosine, and 0.02% (w/v) sodium dodecyl sulfate, at 55° C. for 30 minutes. A sample containing DNA having a sequence complementary to one of the immobilized oligonucleotide probes on the array is applied to the support surface of the fiber and incubated for one hour at 55° C. in a sealed hybridization chamber. The support surface is then washed with 2× SSC containing 0.1% SDS (sodium dodecylsulfate) for 5 minutes at 55° C., after which 50 fmole of a fluorescent-labeled detection probe that is complementary to another sequence on the target DNA is applied to the support surface and incubated for one hour at 55° C. The support surface is then washed with 2× SSC containing 0.1% SDS for 5 minutes at 55° C., followed by a wash with 0.1× SSC, then dried and the fluorescence pattern recorded by the image analysis instrument.

One of skill in the art will appreciate that the present invention can provide a variety of oligonucleotide sequences in association with a particular fiber bundle. In one embodiment, for example, the fiber wells are coated individually with specific binding agents. In another embodiment, the fiber wells are coated simultaneously with a general binding agent (e.g., iminobiotin). In this embodiment, the fiber wells are loaded from a mixture of specific binding agent micro-beads each containing the complement to the general binding agent (e.g., x-avidin).

Optionally, the system further includes an image analyzer system, e.g., a CCD camera with associated software. The resultant assay array can be used in a conventional manner, e.g., the assay array can be read by a standard array reader, while the master array can be re-used to prepare more assay arrays by repeating some or all of the steps set forth above.

It will be apparent to one of skill in the art that the invention can be applied to any molecular species involved in molecular recognition. While the discussion has provided details regarding nucleic acid applications of the invention, it is understood that the invention is not so limited.

All patents, patent applications, provisional applications, and publications referred to or cited herein are incorporated by reference in their entirety to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

### Example 1

### Preparation of 4-Benzoylbenzoyl Chloride (BBA-Cl) (Compound I)

4-Benzoylbenzoic acid (BBA), 1.0 kg (4.42 moles), was added to a dry 5 liter Morton flask equipped with reflux condenser and overhead stirrer, followed by the addition of 645 ml (8.84 moles) of thionyl chloride and 725 ml of toluene. Dimethylformamide, 3.5 ml, was then added and the mixture was heated at reflux for four (4) hours. After cooling, the solvents were removed under reduced pressure and the residual thionyl chloride was removed by three evaporations using 3×500 ml of toluene. The product was recrystallized from 1:4 toluene:hexane to give 988 g (91% yield) after drying in a vacuum oven. Product melting point was 92-94° C. Nuclear magnetic resonance (NMR) analysis at 80 MHz (¹H NMR (CDCl₃)) was consistent with the desired product: aromatic protons 7.20-8.25 (m, 9H). All chemical shift values are in ppm downfield from a tetramethylsilane internal standard. The final compound was stored for use in the preparation of a monomer used in the synthesis of photoactivatable polymers as described, for instance, in Example 3.

### Example 2

### Preparation of N-(3-Aminopropyl)methacrylamide Hydrochloride (APMA) (Compound II)

A solution of 1,3 diaminopropane, 1910 g (25.77 moles), in 1000 ml of CH₂Cl₂ was added to a 12 liter Morton flask and cooled on an ice bath. A solution of t-butyl phenyl carbonate, 1000 g (5.15 moles), 250 ml of CH₂Cl₂ was then added dropwise at a rate which kept the reaction temperature below 15° C. Following the addition, the mixture was warmed to room temperature and stirred two (2) hours. The reaction mixture was diluted with 900 ml of CH₂Cl₂ and 500 g of ice, followed by the slow addition of 2500 ml of 2.2 N NaOH. After testing to insure the solution was basic, the product was transferred to a separatory funnel and the organic layer was removed and set aside as extract #1. The aqueous fraction was then extracted with 3× 1250 ml of CH₂Cl₂, keeping each extraction as a separate fraction. The four organic extracts were then washed successively with a single 1250 ml portion of 0.6 N NaOH beginning with fraction #1 and proceeding through fraction #4. This wash procedure was repeated a second time with a fresh 1250 ml portion of 0.6 N NaOH. The organic extracts were then combined and dried over Na₂SO₄. Filtration and evaporation of solvent to a constant weight gave 825 g of N-mono-t-butoxycarbonyl- 1,3-diaminopropane which was used without further purification.

A solution of methacrylic anhydride, 806 g (5.23 moles), in 1020 ml of CHCl₃ was placed in a 12 liter Morton flask equipped with overhead stirrer and cooled on an ice bath. Phenothiazine, 60 mg, was added as an inhibitor, followed by the dropwise addition of N-mono-t-butoxycarbonyl-1,3-diaminopropane, 825 g (4.73 moles), in 825 ml of CHCl₃. The rate of addition was controlled to keep the reaction temperature below 10° C. at all times. After the addition was complete, the ice bath was removed and the mixture was left to stir overnight. The product was diluted with 2400 ml of water and transferred to a separatory funnel. After thorough mixing, the aqueous layer was removed and the organic layer was washed-with 2400 ml of 2 N NaOH, insuring that the aqueous layer was basic. The organic layer was then dried over Na₂SO₄ and filtered to remove drying agent. A portion of the CHCl₃ solvent was removed under reduced pressure until the combined weight of the product and solvent was approximately 3000 g. The desired product was then precipitated by slow addition of 11.0 liters of hexane to-the stirred CHCl₃ solution, followed by overnight storage at 4° C. The product was isolated by filtration and the solid was rinsed twice with a solvent combination of 900 ml of hexane and 150 ml of CHCl₃.

Thorough drying of the solid gave 900 g of N-[N′-(t-butyloxycarbonyl)-3-aminopropyl]-methacrylamide, melting point 85.8° C. by DSC. Analysis on an NMR spectrometer was consistent with the desired product: ¹H NMR (CDCl₃) amide NH's 6.30-6.80, 4.55-5.10 (m, 2H), vinyl protons 5.65, 5.20 (m, 2H), methylenes adjacent to N 2.90-3.45 (m, 4H), methyl 1.95 (m, 3H), remaining methylene 1.50-1.90 (m, 2H), and t-butyl 1.40 (s, 9H).

A 3-neck, 2 liter round bottom flask was equipped with an overhead stirrer and gas sparge tube. Methanol, 700 ml, was added to the flask and cooled on an ice bath. While stirring, HCl gas was bubbled into the solvent at a rate of approximately 5 liters/minute for a total of 40 minutes. The molarity of the final HCl/MeOH solution was determined to be 8.5 M by titration with 1 N NaOH using phenolphthalein as an indicator. The N-[N′-(t-butyloxycarbonyl)-3-aminopropyl]methacrylamide, 900 g (3.71 moles), was added to a 5 liter Morton flask equipped with an overhead stirrer and gas outlet adapter, followed by the addition of 1150 ml of methanol solvent. Some solids remained in the flask with this solvent volume. Phenothiazine, 30 mg, was added as an inhibitor, followed by the addition of 655 ml (5.57 moles) of the 8.5 M HCl/MeOH solution. The solids slowly dissolved with the evolution of gas but the reaction was not exothermic. The mixture was stirred overnight at room temperature to insure complete reaction. Any solids were then removed by filtration and an additional 30 mg of phenothiazine were added. The solvent was then stripped under reduced pressure and the resulting solid residue was azeotroped with 3×1000 ml of isopropanol with evaporation under reduced pressure. Finally, the product was dissolved in 2000 ml of refluxing isopropanol and 4000 ml of ethyl acetate were added slowly with stirring. The mixture was allowed to cool slowly and was stored at 4° C. overnight. Compound II was isolated by filtration and was dried to constant weight, giving a yield of 630 g with a melting point of 124.7° C. by DSC. Analysis on an NMR spectrometer was consistent with the desired product: ¹H NMR (D₂O) vinyl protons 5.60, 5.30 (m, 2H), methylene adjacent to amide N 3.30 (t, 2H), methylene adjacent to amine N 2.95 (t, 2H), methyl 1.90 (m, 3H), and remaining methylene 1.65-2.10 (m, 2H). The final compound was stored for use in the preparation of a monomer used in the synthesis of photoactivatable polymers as described, for instance, in Example 3.

### Example 3

### Preparation of N-[3-(4-Benzoylbenzamido)propyl]methacrylamide (BBA-AMPA) (Compound III)

Compound II 120 g (0.672 moles), prepared according to the general method described in Example 2, was added to a dry 2 liter, three-neck round bottom flask equipped with an overhead stirrer. Phenothiazine, 23-25 mg, was added as an inhibitor, followed by 800 ml of chloroform. The suspension was cooled below 10° C. on an ice bath and 172.5 g (0.705 moles) of Compound I, prepared according to the general method described in Example 1, were added as a solid. Triethylamine, 207 ml (1.485 moles), in 50 ml of chloroform was then added dropwise over a 1-1.5 hour time period. The ice bath was removed and stirring at ambient temperature was continued for 2.5 hours. The product was then washed with 600 ml of 0.3 N HCl and 2×300 ml of 0.07 N HCl. After drying over sodium sulfate, the chloroform was removed under reduced pressure and the product was recrystallized twice from 4:1 toluene:chloroform using 23-25 mg of phenothiazine in each recrystallization to prevent polymerization. Typical yields of Compound III were 90% with a melting point of 147-151 ° C. Analysis on an NMR spectrometer was consistent with the desired product: ¹H NMR (CDCl₃) aromatic protons 7.20-7.95 (m, 9H), amide NH 6.55 (broad t, 1H), vinyl protons 5.65, 5.25 (m, 2H), methylene adjacent to amide N's 3.20-3.60 (m, 4H), methyl 1.95 (s, 3H), and remaining methylene 1.50-2.00 (m, 2H). The final compound was stored for use in the synthesis of photoactivatable polymers as described, for instance, in Example 6.

### Example 4

### Preparation of N-Succinimidyl 6-Maleimidohexanoate (MAL-EAC-NOS) (Compound IV)

A functionalized monomer was prepared in the following manner, and was used as described in Example 6 to introduce activated ester groups on the backbone of a polymer. 6-Aminohexanoic acid, 100 g (0.762 moles), was dissolved in 300 ml of acetic acid in a three-neck, 3 liter flask equipped with an overhead stirrer and drying tube. Maleic anhydride, 78.5 g (0.801 moles), was dissoved in 200 ml of acetic acid and added to the 6-aminohexanoic acid solution. The mixture was stirred one hour while heating on a boiling water bath, resulting in the formation of a white solid. After cooling overnight at room temperature, the solid was collected by filtration and rinsed with 2×50 ml of hexane. After drying, the typical yield of the (Z)-4-oxo-5-aza-2-undecendioic acid was 158-165 g (90-95%) with a melting point of 160-165° C. Analysis on an NMR spectrometer was consistent with the desired product: ¹H NMR (DMSO-d₆) amide proton 8.65-9.05 (m, 1H), vinyl protons 6.10, 6.30 (d, 2H), methylene adjacent to nitrogen 2.85-3.25 (m, 2H), methylene adjacent to carbonyl 2.15 (t, 2H), and remaining methylenes 1.00-1.75 (m, 6H).

(Z)-4-Oxo-5-aza-2-undecendioic acid, 150.0 g (0.654 moles), acetic anhydride, 68 ml (73.5 g, 0.721 moles), and phenothiazine, 500 mg, were added to a 2 liter three-neck round bottom flask equipped with an overhead stirrer. Triethylamine, 91 ml (0.653 moes), and 600 ml of tetrahydrofuran (THF) were added and the mixture was heated to reflux while stirring. After a total of four (4) hours of reflux, the dark mixture was cooled to less than 60° C. and poured into a solution of 250 ml of 12 N HCl in 3 liters of water. The mixture was stirred three (3) hours at room temperature and then was filtered through a filtration pad (Celite 545, J. T. Baker, Jackson, Tenn.) to remove solids. The filtrate was extracted with 4×500 ml of chloroform and the combined extracts were dried over sodium sulfate. After adding 15 mg of phenothiazine to prevent polymerization, the solvent was removed under reduced pressure. The 6-maleimidohexanoic acid was recrystallized from 2:1 hexane:chloroform to give typical yields of 76-83 (55-60%) with a melting point of 81-85° C. Analysis on an NMR spectrometer was consistent with the desired product: ¹H NMR (CDCl₃) maleimide protons 6.55 (s, 2H), methylene adjacent to nitrogen 3.40 (t, 2H), methylene adjacent to carbonyl 2.30 (t, 2H), and remaining methylenes 1.05-1.85 (m, 6H).

The 6-maleimidohexanoic acid, 20.0 g (94.7 mmol), was dissolved in 100 ml of chloroform under an argon atmosphere, followed by the addition of 41 ml (0.47 mol) of oxalyl chloride. After stirring for 2 hours at room temperature, the solvent was removed under reduced pressure with 4×25 ml of additional chloroform used to remove the last of the excess oxalyl chloride. The acid chloride was dissolved in 100 ml of chloroform, followed by the addition of 12 g (0.104 mol) of N-hydroxysuccinimide and 16 ml (0.114 mol) of triethylamine. After stirring overnight at room temperature, the product was washed with 4×100 ml of water and dried over sodium sulfate. Removal of solvent gave 24 g of product (82%) which was used without further purification. Analysis on an NMR spectrometer was consistent with the desired product: ¹H NMR (CDCl₃) maleimide protons 6.60 (s, 2H), methylene adjacent to nitrogen 3.45 (t, 2H), succinimidyl protons 2.80 (s, 4H), methylene adjacent to carbonyl 2.55 (t, 2H), and remaining methylenes 1.15-2.00 (m, 6H). The final compound was stored for use in the synthesis of photoactivatable polymers as described, for instance, in Example 6.

### Example 5

### Preparation of Functionalized Monomer: N-Succinimidyl 6-Methacrylamidohexanoate (MA-EAC-NOS) (Compound V)

A functionalized monomer was prepared in the following manner, and was used to introduce activated ester groups on the backbone of a polymer.

6-Aminocaproic acid, 4.00 g (30.5 mmol), was placed in a dry round bottom flask equipped with a drying tube. Methacrylic anhydride, 5.16 g (33.5 mmol), was then added and the mixture was stirred at room temperature for four (4) hours. The resulting thick oil was triturated three times with hexane and the remaining oil was dissolved in chloroform, followed by drying over sodium sulfate.

After filtration and evaporation, a portion of the product was purified by silica gel flash chromatography using a 10% methanol in chloroform solvent system. The appropriate fractions were combined, 1 mg of phenothiazine was added, and the solvent was removed under reduced pressure. Analysis on an NMR spectrometer was consistent with the desired product: ¹H NMR (CDCl₃) carboxylic acid proton 7.80-8.20 (b, 1H), amide proton 5.80-6.25 (b, 1H), vinyl protons 5.20 and 5.50 (m, 2H), methylene adjacent to nitrogen 3.00-3.45 (m, 2H), methylene adjacent to carbonyl 2.30 (t, 2H), methyl group 1.95 (m, 3H), and remaining methylenes 1.10-1.90 (m, 6H).

6-Methacrylamidohexanoic acid, 3.03 g (15.2 mmol), was dissolved in 30 ml of dry chloroform, followed by the addition of 1.92 g (16.7 mmol) of N-hydroxysuccinimde and 6.26 g (30.4 mmol) of 1,3-dicyclohexylcarbodiimide. The reaction was stirred under a dry atmosphere overnight at room temperature. The solid was then removed by filtration and a portion was purified by silica gel flash chromatography. Non-polar impurities were removed using a chloroform solvent, followed by elution of the desired product using a 10% tetrahydrofuran in chloroform solvent. The appropriate fractions were pooled, 0.2 mg of phenothiazine were added, and the solvent was evaporated under reduced pressure. This product, containing small amounts of 1,3-dicyclohexylurea as an impurity, was used without further purification. Analysis on an NMR spectrometer was consistent with the desired product: ¹H NMR (CDCl₃) amide proton 5.60-6.10 (b, 1H), vinyl protons 5.20 and 5.50 (m, 2H), methylene adjacent to nitrogen 3.05-3.40 (m, 2H), succinimidyl protons 2.80 (s, 4H), methylene adjacent to carbonyl 2.55 (t, 2H), methyl 1.90 (m, 3H), and remaining methylenes 1.10-1.90 (m, 6H). The final compound was stored for use in the synthesis of photoactivatable polymers as described herein.

### Example 6

### Preparation of Copolymer of Acrylamide, BBA-APMA, and MAL-EAC-NOS (Photo PA-PolyNOS)

A photoactivatable copolymer of the present invention was prepared in the following manner. Acrylamide, 4.298 g (60.5 mmol), was dissolved in 57.8 ml of tetrahydrofuran, followed by 0.219 g (0.63 mmol) of Compound III, prepared according to the general method described in Example 3, 0.483 g (1.57 mmol) of Compound IV, prepared according to the general method described in Example 4, 0.058 ml (0.39 mmol) of N,N,N′,N′-tetramethylethylenediamine (TEMED), and 0.154 g (0.94 mmol) of 2,2′-azobisisobutyronitrile (AIBN). The solution was deoxygenated with a helium sparge for 3 minutes, followed by an argon sparge for an additional 3 minutes. The sealed vessel was then heated overnight at 60° C. to complete the polymerization. The solid product was isolated by filtration and the filter cake was rinsed thoroughly with THF and CHCl₃. The product was dried in a vacuum oven at 30° C. to give 5.34 g of a white solid. NMR analysis (DMSO-d₆) confirmed the presence of the N-oxysuccinimide (NOS) group at 2.75 ppm and the photogroup load was determined to be 0.118 mmol BBA/g of polymer. The MAL-EAC-NOS composed 2.5 mole % of the polymerizable monomers in this reaction to give Compound VI-A.

The above procedure was used to prepare a polymer having 5 mole % Compound IV. Acrylamide, 3.849 g (54.1 mmol), was dissolved in 52.9 ml of THF, followed by 0.213 g (0.61 mmol) of Compound III, prepared according to the general method described in Example 3, 0.938 g (3.04 mmol) of Compound IV, prepared according to the general method described in Example 4, 0.053 ml (0.35 mmol) of TEMED and 0.142 g (0.86 mmol) of AIBN. The resulting solid, Compound VI-B, when isolated as described above, gave 4.935 g of product with a photogroup load of 0.101 mmol BBA/g of polymer.

The above procedure was used to prepare a polymer having 10 mole % of Compound IV. Acrylamide, 3.241 g (45.6 mmol), was dissolved in 46.4 ml of THF, followed by 0.179 g (0.51 mmol) of Compound III, prepared according to the general method described in Example 3, 1.579 g (5.12 mmol) of Compound IV, prepared according to the general method described in example 4, 0.047 ml (0.31 mmol) of TEMED and 0.126 g (0.77 mmol) of AIBN. The resulting solid, Compound VI-C, when isolated as described above, gave 4.758 g of product with a photogroup load of 0.098 mmol BBA/g of polymer.

A procedure similar to the above procedure was used to prepare a polymer having 2.5 mole % Compound IV and 2 mole % Compound III. Acrylamide, 16.43 g (231.5 mmol); Compound III, prepared according to the general method described in Example 3, 1.70 g (4.85 mmol); Compound IV, prepared according to the general method described in Example 4, 1.87 g (6.06 mmol); and THF (222 ml) were stirred in a round bottom flask with an argon sparge at room temperature for 15 minutes. TEMED, 0.24 ml (2.14 mmol), and AIBN, 0.58 g (3.51 mmol), were added to the reaction. The reaction was then refluxed for four (4) hours under an atmosphere of argon. The resulting solid, Compound VI-D, when isolated as described above, gave 19.4 g of product with a photogroup load of 0.23 mmol BBA/g of polymer.

### Example 7

### Synthesis of Multi-Ligand Conjugate

A multi-ligand conjugate is prepared as follows. A 200 μl aliquot of polystyrene microbeads (1 μm diameter) (obtained from Prolabo, Bangs Labs, Carmel, Ind., product number K100), containing 10% by weight microbeads in aqueous suspension, are mixed with a solution of Photo-PA-PolyNOS (prepared as described in Example 6 above) containing 1.0 mg of polymer in 200 μl of deionized water. The solution is mixed for one hour at room temperature.

After mixing for one hour at room temperature, the suspension is illuminated for two minutes while mixing, using a Dymax lamp (25 mjoule/cm² as measured at 335 nm with a 10 nm band pass filter on an International Light radiometer). The microbeads are then washed twice with 2 ml of deionized water by centrifugation at 15,000 rpm for 15 minutes and resuspension in phosphate buffer solution (PBS, pH 7.5).

The washed beads are then incubated with a solution containing a mixture of two oligonucleotide sequences, each modified to contain a primary amine at the 3′ end, and biocytin (10 μM) in 0.05M phosphate buffer, pH 7.5. The first oligonucleotide sequence is the complement to the known sequence of a specific address oligonucleotide provided on a master array. The first oligonucleotide is provided as a 20-mer, at 5 μM. The first oligonucleotide is synthesized by first determining the sequence of a specific address oligonucleotide to be used in the invention. The complement to the address oligonucleotide is then determined using Watson-Crick base pairing rules. Once the complementary sequence is determined, the first oligonucleotide is synthesized using standard nucleic acid synthesis techniques, for example, solid phase synthesis.

The second oligonucleotide sequence is an analytical sequence that is complementary to a known sequence of a target nucleic acid suspected to be present in a test sample. The second oligonucleotide is provided as a 30 mer, at 10 μM. The second oligonucleotide is synthesized by first determining the sequence of a specific target ligand to be detected using the probe array of the invention. Tthe complement to the target ligand is determined using Watson-Crick base pairing rules. Once the complementary sequence is determined, the second oligonucleotide is synthesized using standard nucleic acid synthesis techniques.

The microbead suspension is incubated with the oligonucleotides overnight at room temperature. After overnight incubation, the beads are washed twice with 1 ml aliquots of phosphate buffer solution (PBS, pH 7.5), then stored at 4° C. until used. A series of multi-ligand conjugates, each with unique address and analytical sequences is made in this manner for replicating probe arrays.

### Example 8

### Synthesis of a Spaced Epoxide Monomer (Compound VII)

To three ml of chloroform was added isocyanatoethylmethacrylate (1.0 ml, 7.04 mmol), glycidol (0.50 ml, 7.51 mmol) and triethylamine (50 μl, 0.27 mmol). The reaction was stirred at room temperature overnight. The product was purified on a silica gel column and the structure confirmed by NMR. The yield was 293 mg (18% yield).

### Example 9

### Preparation of Copolymer of Acrylamide, BBA-APMA, and Glycidyl Methacrylate (Photo PA-Polyepoxide) (Compound VIII)

Acrylamide (7.1 g, 99.35 mmol), BBA—APMA (0.414 g, 1.18 mmol) and 2,2′-azobis(2-methylbutyronitrile) (“VAZO 67”, 0.262 g, 1.4 mmol) were dissolved in 108 ml of THF. To this solution was added 2.4 ml of glycidylmethacrylate (17.7 mmol). The solution was sparged with helium for four minutes, then with argon for four minutes, then capped tightly and heated at 61° C. overnight while mixing. The polymer was collected by filtration, then suspended in methanol and mixed, after which it was again collected by filtration and washed with chloroform, then dried in a vacuum oven at 30 ° C.

### Example 10

### Preparation of Microscope Slides Coated with PolyEpoxide

Soda lime glass microscope slides (Erie Scientific, Portsmouth, N.H.) were silane treated by dipping in a mixture of p-tolyldimethylchlorosilane (T-silane) and N-decyldimethylchlorosilane (D-Silane, United Chemical Technologies, Bristol, Pa.), 1% each in acetone, for 1 minute. After air drying, the slides were cured in an oven at 120° C. for one hour. The slides were then washed with acetone followed by distilled water dipping. The slides were further dried in an oven for 5-10 minutes.

Compound VIII (Example 9) was sprayed onto the silane treated slides, which were then illuminated using a Dymax lamp (25 mjoule/cm² as measured at 335 nm with a 10 nm band pass filter on an International Light radiometer) while wet, washed with water, and dried.

### Example 11

### Preparation of Microarrays with Photo-polyepoxide Coated Slides

Oligonucleotides, either aminated or nonaminated, were printed onto slides at 8 μM in 0.15 M phosphate buffer using an X, Y, Z motion controller to position ChipMaker 2 microarray spotting pins (Telechem International). The slides were either coated with photo-PA-polyepoxide as in Example 10, with photo-PA-PolyNOS by the same procedure or with polylysine by published methods (See U.S. Pat. No. 5,087,522, Brown et al., “Methods for Fabricating Microarrays of Biological Samples,” and the references cited therein).

The printed epoxide and NOS slides were incubated overnight at room temperature and 75% relative humidity. The printed polylysine slides were processed by the published procedure. The slides were scanned to measure the Cy3 fluorescence of immobilized capture oligonucleotides, then hybridized at 41° C. overnight with Cy5-labeled oligonucleotide (1 pmole/slide) that was complementary to the capture oligonucleotides. The slides were scanned with a laser scanner to measure the fluorescence intensities of the hybridized oligonucleotides. Because the amount of capture oligonucleotide immobilized with the amine-silane slides was low, they were not hybridized.

TABLE 1
Compounds.

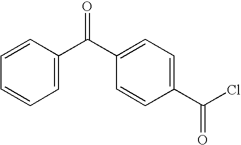

COMPOUND I

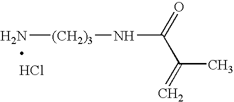

COMPOUND II

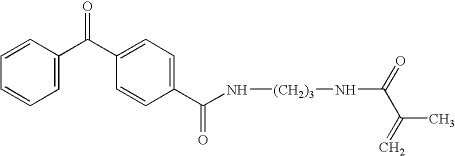

COMPOUND III

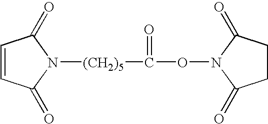

COMPOUND IV

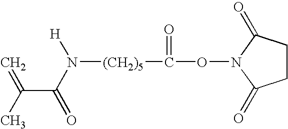

COMPOUND V

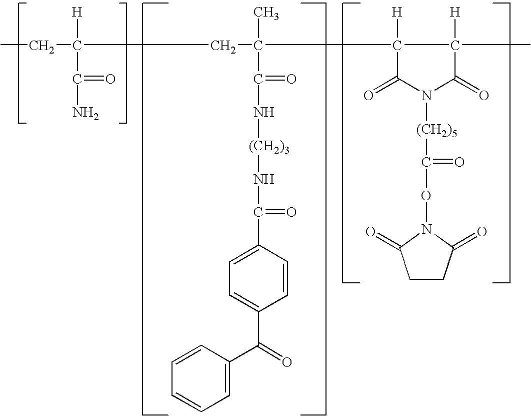

COMPOUND VI

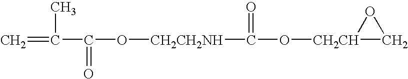

COMPOUND VII

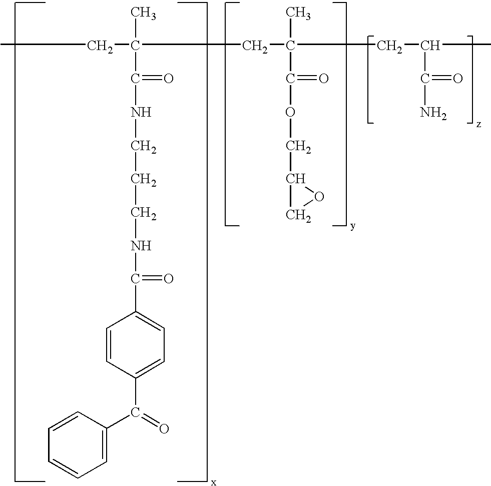

COMPOUND VIII
(where x = 0.1 to 5 mole %, y = 2 to 30 mole %, and z = 65 to 97.9 mole %)

(where x=0.1 to 5 mole %, y=2 to 3 mole %,and z=65 to 97.9 mole %)

While a preferred embodiment of the present invention has been described, it should be understood that various changes, adaptations and modifications may be made therein without departing from the spirit of the invention and the scope of the appended claims.

What is claimed is:

1. A system for replicating a specific binding ligand probe array, the system comprising:
a) a master array comprising a support surface having a plurality of address ligands immobilized thereon;
b) a plurality of multi-ligand conjugates, each multi-ligand conjugate comprising: (i) a core; (ii) at least one molecule of a first ligand binding domain, comprising a ligand selected to bind in a complementary manner with a specific address ligand of the master array, (iii) at least one molecule of a second ligand binding domain, comprising a ligand selected to bind in a complementary manner with a target ligand, and (iv) at least one molecule of a third ligand, wherein the first ligand binding domain, the second ligand binding domain, and the third ligand are attached to the core; and
c) an assay array support comprising a support surface for the replicate array.

2. The system according to claim 1 wherein the address ligands, and the first and second binding ligands each, independently, comprise a nucleic acid.

3. The system according to claim 1 further comprising a linking agent, wherein the linking agent is attached to the address ligand and to the master array support surface.

4. The system according to claim 3 wherein the linking agent comprises a micro-bead.

5. The system according to claim 1 wherein the first ligand binding domain, second ligand binding domain and third ligand are independently attached to the core of the multi-ligand conjugate.

6. The system according to claim 1 wherein the third ligand comprises a biotin derivative.

7. The system according to claim 1 wherein the third ligand comprises polymerizable groups.

8. The system according to claim 7 wherein the polymerizable groups are selected from the group consisting of acrylic groups and vinyl groups.

9. The system according to claim 1 wherein the assay array support further comprises attachment sites for the third ligand.

10. The system according to claim 9 wherein the third ligand comprises a binding ligand, and the attachment sites comprise molecules of a binding partner specific for the third ligand.

11. A method for replicating a specific binding ligand probe array, the method comprising:
a) providing a master array comprising a support surface having a plurality of address ligands immobilized thereon;
b) providing a plurality of multi-ligand conjugates, each multi-ligand conjugate comprising: (i) a core; (ii) at least one molecule of a first ligand binding domain, comprising a ligand selected to bind in a complementary manner with a specific address ligand of the master array, (iii) at least one molecule of a second ligand binding domain, comprising a ligand selected to bind in a complementary manner with a target ligand, and (iv) at least one molecule of a third ligand, wherein the first ligand binding domain, the second ligand binding domain, and the third ligand are attached to the core;
c) attaching the multi-ligand conjugates to the master array by allowing the first ligand binding domains to bind complementary address ligands;
d) providing an assay array support comprising a support surface for the replicate array;
e) bringing the assay array support into sufficient proximity with the master array, under conditions suitable to permit the attached multi-ligand conjugates to attach to the assay array support; and
f) disassociating the bound complementary address ligand and first ligand binding domain under conditions suitable to permit the assay array support to be recovered and used.

12. The method according to claim 11, wherein the third ligand comprises a biotin derivative.

13. The method according to claim 11, wherein the third ligand comprises a polymerizable group, the method further comprising the step of reacting the third ligand to form a polymer film.

14. The method according to claim 13, wherein the polymerizable group is selected from vinyl groups and acrylic groups.

15. The method according to claim 11 wherein the address ligands, and the first and second binding ligands each, independently, comprise a nucleic acid.

16. The method according to claim 11 wherein the master array further comprises a linking agent, wherein the linking agent is attached to the address ligand and to the master array support surface.

17. The method according to claim 16 wherein the linking agent comprises a micro-bead.

18. The method according to claim 11 wherein the assay array support further comprises attachment sites for the third ligand.

19. The method according to claim 18 wherein the third ligand comprises a binding ligand, and the attachment sites comprise molecules of a binding partner specific for the third ligand.

20. A system for replicating a specific binding ligand probe array, the system comprising:
a) a master array comprising a support surface having a plurality of address ligands immobilized thereon;
b) a plurality of multi-ligand conjugates- each multi-ligand conjugate comprising: (i) a core; (ii) at least one molecule of a second ligand binding domain, comprising a ligand selected to bind in a complementary manner to a specific target ligand and to a specific address ligand, and (iii) at least one molecule of a third ligand, wherein the second ligand binding domain, and the third ligand are attached to the core; and
c) an assay array support comprising a support surface for the replicate array.

21. The system according to claim 20 wherein the address ligands, and the first and second binding ligands each, independently comprise a nucleic acid.

22. The system according to claim 20 wherein the third ligand comprises a biotin derivative.

23. The system according to claim 20 wherein the third ligand is selected from the group of binding ligands and polymerizable groups.

24. The system according to claim 23 wherein the polymerizable groups are selected from vinyl groups and acrylic groups.

25. A system for preparing a replicable array, in the form of a reusable array, the system comprising:
a) a master array comprising a plurality of optical fibers, each optical fiber having a support surface located at the distal end of the fiber; and
b) a plurality of oligonucleotide binding domains, each oligonucleotide binding domain comprising an oligonucleotide sequence selected to bind in a specific manner with a target ligand.
